(19) United States
(12) Patent Application Publication
(10) Pub. No.: US 2026/0224595 A1
(43) Pub. Date: Aug. 6, 2026

(54) COMPOUNDS FOR THE TREATMENT OF MYOTONIC DYSTROPHY

(71) Applicant: ETH ZURICH, Zurich (CH)

(72) Inventors: Jonathan HALL, Dornach (CH); Alok Kumar BEHERA, Zurich (CH)

(21) Appl. No.: 19/117,798

(22) PCT Filed: Oct. 3, 2023
(86) PCT No.: PCT/EP2023/077313

§ 371 Date: Apr. 2, 2025

(30) Foreign Application Priority Data
Oct. 4, 2022 (EP) 22199499.9

(51) Int. Cl. A61K 31/635; A61K 31/5025; A61P 21/00

(52) U.S. Cl. CPC A61K 31/635; A61K 31/5025; A61P 21/00

(57) ABSTRACT

The present invention relates to compositions for use in the treatment, amelioration, and or prevention of myotonic dystrophy (DM) including DM1 and DM2. The compounds can disrupt the activity of Lin28 (e.g., Lin28A and/or Lin28B), RNA binding proteins responsible for inhibiting the maturation of miRNA precursors to mature miRNA. Upon treatment with the inventive compounds, the DM1 and DM2 cells exhibited (i) increased MEF2A expression (i.e., de-repression of MEF2A associated with DM); (ii) decreased CELF protein expression (e.g., decreased CELF1 expression); (iii) increased expression of miRNAs associated with DM and shifted the targetomes of the miRNAs toward a healthy signature; (iv) decreased expression of sodium-, potassium- and calcium ion channels with roles in myotonia, muscle wasting, and cardiac conduction; (v) increased expression of CLCN1; and (vi) decreased expression of PKC.

The present invention relates to compounds for the treatment, amelioration, and/or prevention of myotonic dystrophy (DM) in a subject in need thereof. The inventive compounds can inhibit Lin28 and its interaction with precursors of certain miRNAs that help regulate the expression of ion channels involved in DM in cells derived from DM patients. The inventive compounds restored the activity of the miRNAs and decreased the expression of the ion channels regulated by the miRNAs. Additionally, the inventive compounds increased expression of the transcription factor MEF2 (e.g., MEF2A) and decreased expression of CELF proteins (e.g., CELF1), increased expression of CLCN1, and decreased expression of PKC.

### RELATED ART

Myotonic dystrophy (DM) is a type of muscular dystrophy that causes multiple physiological limitations, including progressive muscle loss and weakness, muscle myotonia, cardiac defects, and possible death. There are two major types of DM, myotonic dystrophy type 1 (DM1) and myotonic dystrophy type 2 (DM2) (Thornton, Neurol Clin, 2014, 32:705-719; Meola et al, Neurol Sci, 2017, 38:535-546; Lanni et al., Neurobiol Dis, 2019, 132:104533; Meola, Acta Myol 2020, 39:222-234). Both are caused by non-coding intragenic repeat tract expansions of (i) CTG in the DMPK gene (DM1); or (ii) CCTG in the CNBP1 gene (DM2). CTG repeats in the DMPK gene are pathogenic above 50 repeats, and CCTG repeats in the CNBP1 gene are pathogenic above 75 repeats. When the DMPK or CNBP1 genes are transcribed, the CUG^{epx}/CCUG^{exp} repeats in the encoded mRNAs form hairpin structures that sequester proteins of the muscle blind-like protein family (MBNL1-2), causing a loss-of-function of these proteins. The pathologic repeats also cause gain-of-function mis-regulation of CUGBP Elav-Like Family Member 1 (CELF1) proteins. Together, the de-regulation of MBNL1-2 and CELF1 can result in improper splicing of hundreds of genes, resulting in the phenotypes characteristic of DM (see Lopez Castel et al., Int. J. Mol. Sci., 2019, 20, 5600).

There is currently no cure or specific treatment for myotonic dystrophy (e.g., with conventional small molecule drugs). Since it is a multisystemic disorder, it affects many organs such as the eye (e.g., leading to cataracts); the heart (e.g., leading to cardiac abnormalities); and the gastrointestinal system (e.g., leading to gastrointestinal dysfunction). Potential treatments for myotonia include mexiletine, quinine, phenytoin, and other anticonvulsant drugs (e.g., antiepileptic drugs). Other drugs including phenytoin, procainamide, propafenone, flecainide, and carbamazepine have been evaluated as potential treatments for myotonia, however, there is not enough information yet from clinical studies to conclude that they are effective.

Non-pharmaceutical therapy can mitigate the effects of DM. Ankle supports and leg braces can help aid walking as muscle weakness worsens. Physical therapy and other rehabilitative measures can help improve muscle function. Skeletal muscle weakness can be reduced by ankle foot orthosis (AFO), and scooters or wheelchairs can be used as walking aids. However, none of these treatments addresses the underlying cause of muscle weakness brought on by DM.

Pharmaceutical therapies have been proposed for the treatment of DM, including drugs that upregulate the activity of MEF transcription factors, drugs that inhibit CELF proteins, and drugs that release MBNL from the binding of CUG^{exp}. Such treatments include small molecules, anti-miRNA oligonucleotides, and/or peptide therapies (Jahromi et al., ACS Chem. Biol., 2013, 8, 1037-1043; Angelbello et al., Cell Chem. Biol., 2021, 28, 34-45; Warf et al., PNAS, 2009, 106(44), 18551-18556; Cerro-Herreros et al., Mol. Ther. Nuc. Acid., 2020, 21, 837-849).

Rau et al. (Nat. Struct. Mol. Biol., 2011, 18(7):840-845) teaches that MBNL1 binds to a UGC motif located within the loop of pre-miR-1 and competes for the binding of Lin28, which promotes pre-miR-1 uridylation by ZCCHC11 (TUT4) and blocks Dicer processing in heart samples from patients with DM.

Roos et al. (ACS Chem. Biol., 2016, 11, 2773-2781) teaches small molecules capable of blocking the interaction between Lin28 and pre-let-7 in cancer cells.

Hall (Wansink et al., Neuromuscular Disorders, 2020, 30, 521-531) teaches a FRET-based high-throughput screen that identified drug-like compounds that inhibit the binding of Lin28 to let-7 miRNA precursors.

There is currently no known treatment for DM using conventional small molecule drugs. Accordingly, there is a need for therapies that can alleviate and/or reverse the effects of DM in subjects.

Camerino et al. (Nat. Sci. Rep., 2019, 9, 3198) teaches that expression of ClC-1 chloride channel (ClCN1) was reduced in an animal model of Amyotrophic Lateral Sclerosis (ALS), and the expression of Protein Kinase-C (PKC), known to control ClC-1 activity, was increased, causing inhibition of ClC-1 in the animal model.

Sahadevan et al., (Nat. Comm., 2021, 12, 3027) and Wang et al., (J. Appl. Physiol., 2012, 113, 1902-1920) teach that expression of the ion channel ATP1B is increased in models of ALS.

Miranda et al., (J. Gen. Physiol., 2017, 149(1), 55-74) teaches that ClC-1 is reduced in a mouse model of Huntington's Disease (HD), and the ClC-1 loss-of-function correlated with increased aberrant mRNA processing and decreased levels of full-length ClC-1 mRNA. Waters et al., (PNAS, 2013, 110(22), 9160-9165) teaches that the expression of ClC-1 in Huntington disease muscle was compromised by improper splicing and a corresponding reduction in total CLCN1 (gene for ClC-1) mRNA.

Weeks and McMullen (Circ. Genom. Precis. Med. 2018; 11:e002104) teach that multiple studies suggest that inhibition of PKCα in the failing heart would be beneficial. Weeks and McMullen further teach that the development of PKC-specific inhibitors, including for cardiac pathogenesis (e.g., heart failure and atherosclerosis) has been of interest, but results from clinical trials have been disappointing.

Zarate and Manji (CNS Drugs. 2009, 23(7): 569-582) teach that a growing body of work both on a preclinical and clinical level indicates that PKC signalling may play an important role in the pathophysiology and treatment of bipolar disorder.

Yuan et al., (Sci. Rep., 2015, 5, 12889) teaches that targeted inhibition of protein kinase C (PKC) inhibits hepatocellular carcinoma (HCC) proliferation and metastasis.

Thai et al., (J. Biol. Chem., 1998, 273(23): 14285-14292) teaches that MEF2 binding activity is necessary for regulation of the GLUT4 gene promoter in muscle and adipose tissue. Physiologic manipulation of GLUT4 gene expression is a target for therapeutic interventions designed to treat the insulin resistance associated with diabetes.

Mora and Pessin (J. Biol. Chem., 2000, 275(21): 16323-16328) teach that MEF2A-MEF2D heterodimer is selectively decreased in insulin-deficient diabetes and is responsible for hormonally regulated expression of the GLUT4 gene. Brozinick et al. (Diabetes, 2001, 50: 593-600) teach that increased expression of GLUT4 in skeletal muscle produces changes (lowered blood glucose, increased insulin, and contractile-stimulated glucose transport) that could be of benefit in the treatment of type 2 diabetes. Gibbs et al. (J. Clin. Invest., 1995, 95(4): 1512-1518) teaches that GLUT4 upregulation overcomes the glucose transporter translocation defect and alleviates insulin resistance in genetically diabetic mice, thus resulting in markedly improved glycemic control.

There is a need for therapies that can alleviate and/or reverse the effects of ALS, HD, heart disease (e.g., atherosclerosis), bipolar, diabetes, and/or cancers (e.g., hepatocellular carcinoma) in subjects.

### SUMMARY OF THE INVENTION

The present invention provides evidence for a unifying mechanism in DM1 and DM2, in which a network of alternative splicing/miRNA cross-talk is coordinated by the over-expression of Lin28. As demonstrated in the Detailed Description below, the inventors have identified inventive small molecule compounds capable of inhibiting Lin28 (e.g., Lin28A and/or Lin28B) and reducing its activity in cells obtained from DM1 and DM2 patients. Upon treatment with the inventive compounds, the DM1 and DM2 cells exhibited (i) increased MEF2A expression (i.e., de-repression of MEF2A associated with DM); (ii) decreased CELF protein expression (e.g., decreased CELF1 expression); (iii) increased expression of miRNAs associated with DM (e.g., let7, miR-9, miR-30, miR-29, miR-181, miR-1 and miR-107) and the targetomes of the miRNAs were shifted toward a healthy signature; (iv) decreased expression of sodium-, potassium- and calcium ion channels with roles in myotonia, muscle wasting, and cardiac conduction (e.g., ATP1B1, SERCA2, KCNJ2, CACNA1S, SERCA1, and CACNA1C); (v) increased expression of CLCN1; and (vi) decreased expression of PKC.

Notably, ATP1B1 (sodium/potassium-transporting ATPase Subunit Beta-1) is involved in DM pathology and regulated by the same miRNAs that are increased by treatment with the inventive small molecules disclosed herein. Treatment with the inventive compounds led to a >75% inhibition of CELF1, SERCA1, SERCA2, CACNA1S, and CACNA1C in DM cells. Without wishing to be bound by theory, treatment of DM1 and DM2 cells with the inventive compounds was consistent with additive concerted inhibition of the miRNAs. Additional features and advantages of the technology will be apparent upon reading the Detailed Description, below.

Thus, in one aspect the present invention relates to compounds for use in the inhibition of an RNA binding protein (RBP) (e.g., Lin28; preferably Lin28A and/or Lin28B) and/or to the de-repression of a transcription factor (e.g., MEF, preferably MEF2A) for the prevention and/or treatment of DM (e.g., DM1 and/or DM2). The invention further relates to compounds for use in the prevention or treatment of DM in patients with DM. Preferably, the present invention relates to compounds for use in the prevention or treatment of DM wherein the compound inhibits Lin28 (e.g., Lin28A and/or Lin28B) activity.

In one aspect, the present invention provides a compound, wherein said compound is Compound I and/or Compound II, or a pharmaceutically acceptable salt, stereoisomer, enantiomer, tautomer, hydrate or solvate thereof, for use in a method of preventing or treating a myotonic dystrophy in a subject:

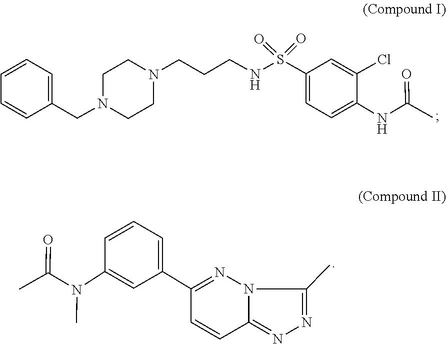

In one aspect, the present invention provides a pharmaceutical composition comprising Compound I and/or Compound II, and a pharmaceutically acceptable carrier or excipient.

### DESCRIPTION OF FIGURES

FIG. 1A is a graph showing reduction in LIN28A levels after 72 h treatment with 0, 30, 60 and 180 μM of Compound II in primary skeletal myotubes from one DM1 patient. Data were collected by Western blot and analyzed by densitometry and were normalized to GAPDH. Fold change relative to control (0 μM of Compound II) and control value is set to 1.0. Error bar indicates mean±SD (n=3 independent experiments). *p<0.05, **p<0.01, ***p<0.001.

FIG. 1B is a graph showing increase in MEF2A concentrations 72 h treatment with 0, 30, 60 and 180 μM of Compound II in in primary skeletal myotubes from one DM1 patient. Data were collected by Western blot and analyzed by densitometry and were normalized to GAPDH. Fold change relative to control (0 μM of Compound II) and control value is set to 1.0. Error bar indicates mean±SD (n=3 independent experiments). *p<0.05, **p<0.01, ***p<0.001.

FIG. 1C is a graph showing reduction in LIN28 protein levels after 72 h treatment with 0, 30, 60 and 180 μM of Compound I in primary skeletal myotubes from one DM1 patient. Data were collected by Western blot and analyzed by densitometry and were normalized to GAPDH. Fold change relative to control (0 μM of Compound I) and control value is set to 1.0. Error bar indicates mean±SD (n=3 independent experiments). *p<0.05, **p<0.01, ***p<0.001.

FIG. 1D is a graph showing increase in MEF2A concentrations 72 h treatment with 0, 30, 60 and 180 μM of Compound I in in primary skeletal myotubes from one DM1 patient. Data were collected by Western blot and analyzed by densitometry and were normalized to GAPDH. Fold change relative to control (0 μM of Compound I) and control value is set to 1.0. Error bar indicates mean±SD (n=3 independent experiments). *p<0.05, **p<0.01, ***p<0.001.

FIG. 2A is a graph showing reduction in LIN28 levels after 72 h treatment with 0, 30, 60 and 180 μM of Compound II in primary skeletal myotubes from one DM2 patient. Data were collected by Western blot and analyzed by densitometry and were normalized to GAPDH. Fold change relative to control (0 μM of Compound II) and control value is set to 1.0. Error bar indicates mean±SD (n=3 independent experiments). *p<0.05, **p<0.01, ***p<0.001.

FIG. 2B is a graph showing increase in MEF2A concentrations 72 h treatment with 0, 30, 60 and 180 μM of Compound II in in primary skeletal myotubes from one DM2 patient. Data were collected by Western blot and analyzed by densitometry and were normalized to GAPDH. Fold change relative to control (0 μM of Compound II) and control value is set to 1.0. Error bar indicates mean±SD (n=3 independent experiments). *p<0.05, **p<0.01, ***p<0.001.

FIG. 3A is a graph showing reduction in LIN28 levels after 72 h treatment with 0, 30, 60 and 180 μM of Compound II in primary skeletal myotubes from Cl5-DM1 cell line. Data were collected by Western blot and analyzed by densitometry and were normalized to GAPDH. Fold change relative to control (0 μM of Compound II) and control value is set to 1.0. Error bar indicates mean±SD (n=3 independent experiments). *p<0.05, **p<0.01, ***p<0.001.

FIG. 3B is a graph showing increase in MEF2A concentrations 72 h treatment with 0, 30, 60 and 180 μM of Compound II in in primary skeletal myotubes from Cl5-DM1 cell line. Data were collected by Western blot and analyzed by densitometry and were normalized to GAPDH. Fold change relative to control (0 μM of Compound II) and control value is set to 1.0. Error bar indicates mean±SD (n=3 independent experiments). *p<0.05, **p<0.01, ***p<0.001.

FIG. 4A is a graph showing the concentration-dependent de-repression of mature miRNAs 20 let7i, miR-1, miR-9, miR-29a, miR-30a, miR-30c miR-107, miR-133, miR-181a, miR-181b, let-7g, and miR-122 relative to the levels of U6 snRNA 72 h after treatment with Compound II at 0, 30, 60 and 180 μM in six DM1 patient myotubes. The concentration of mature miRNAs was measured by real time RT-PCR Taqman assay. Fold changes are measured relative to untreated cells which is set to 1.0.

FIG. 4B is a graph showing the concentration-dependent de-repression of mature miRNAs let7i, miR-9, miR-29a, miR-30a, miR-107, miR-181a, miR-181b, let-7g, and miR-122 relative to the levels of U6 snRNA 72 h after treatment with Compound I at 0, 30, 60 and 180 μM in DM1 patient myotubes. The concentration of mature miRNAs was measured by real time RT-PCR Tagman assay. Fold changes are measured relative to untreated cells which is set to 1.0. Error bar indicates mean±SD (n=2 independent experiments).

FIG. 5A is a series of graphs showing the log₂-fold changes of miRNA-targetomes in DM1 patient-derived cells before treatment with Compound II (dashed line) and after treatment with Compound II (solid line). Each panel shows changes for one miRNA targetome (identified in each panel). The number of expressed miRNA targets is indicated in parenthesis. Median fold changes for each targetome and comparison are marked as a diamond, and indicated on the baseline of each panel. P values were calculated using a Wilcoxon ranksum test and indicate the significance of the difference between the two curves in each panel.

FIG. 5B is a series of graphs showing the log₂-fold changes of miRNA-targetomes in DM1 patient-derived cells after treatment with Compound II (solid line) compared with healthy volunteers (dotted line). Each panel shows changes for one miRNA targetome (identified in each panel). The number of expressed miRNA targets is indicated in parenthesis. Median fold changes for each targetome and comparison are marked as a diamond, and indicated on the baseline of each panel. P values were calculated using a Wilcoxon ranksum test and indicate the significance of the difference between the two curves in each panel.

FIG. 5C is a series of graphs showing the log 2-fold changes of miRNA-targetomes in DM2 patient-derived cells before treatment with Compound II (dashed line) and after treatment with Compound II (solid line). Each panel shows changes for one miRNA targetome (identified in each panel). The number of expressed miRNA targets is indicated in parenthesis. Median fold changes for each targetome and comparison are marked as a diamond, and indicated on the baseline of each panel. P values were calculated using a Wilcoxon ranksum test and indicate the significance of the difference between the two curves in each panel.

FIG. 5D is a series of graphs showing the log₂-fold changes of miRNA-targetomes in DM2 patient-derived cells after treatment with Compound II (solid line) compared with healthy volunteers (dotted line). Each panel shows changes for one miRNA targetome (identified in each panel). The number of expressed miRNA targets is indicated in parenthesis. Median fold changes for each targetome and comparison are marked as a diamond, and indicated on the baseline of each panel. P values were calculated using a Wilcoxon ranksum test and indicate the significance of the difference between the two curves in each panel.

FIG. 6A is a series of graphs showing the log₂-fold changes of miRNA-targetomes in DM1 patient-derived cells before treatment with Compound II (dashed line) and after treatment with Compound II (solid line). Each panel shows changes for one miRNA targetome (identified in each panel). The number of expressed miRNA targets is indicated in parenthesis. Median fold changes for each targetome and comparison are marked as a diamond, and indicated on the baseline of each panel. P values were calculated using a Wilcoxon ranksum test and indicate the significance of the difference between the two curves in each panel.

FIG. 6B is a series of graphs showing the log₂-fold changes of miRNA-targetomes in DM1 patient-derived cells in the absence of treatment (dashed line) compared with healthy volunteers (dotted line). Each panel shows changes for one miRNA targetome (identified in each panel). The number of expressed miRNA targets is indicated in parenthesis. Median fold changes for each targetome and comparison are marked as a diamond, and indicated on the baseline of each panel. P values were calculated using a Wilcoxon ranksum test and indicate the significance of the difference between the two curves in each panel.

FIG. 6C is a series of graphs showing the log₂-fold changes of miRNA-targetomes in DM2 patient-derived cells before treatment with Compound II (dashed line) and after treatment with Compound II (solid line). Each panel shows changes for one miRNA targetome (identified in each panel). The number of expressed miRNA targets is indicated in parenthesis. Median fold changes for each targetome and comparison are marked as a diamond, and indicated on the baseline of each panel. P values were calculated using a Wilcoxon ranksum test and indicate the significance of the difference between the two curves in each panel.

FIG. 6D is a series of graphs showing the log 2-fold changes of miRNA-targetomes in DM2 patient-derived cells in the absence of treatment (dashed line) compared with healthy volunteers (dotted line). Each panel shows changes for one miRNA targetome (identified in each panel). The number of expressed miRNA targets is indicated in parenthesis. Median fold changes for each targetome and comparison are marked as a diamond, and indicated on the baseline of each panel. P values were calculated using a Wilcoxon ranksum test and indicate the significance of the difference between the two curves in each panel.

FIG. 7 is a graph showing reduction of CELF1 expression after transfection of let-7i- and miR-30 mimics in Ctrl-C148 cell lines compared to a mock transfected cells. Data were collected by Western blot and analyzed by densitometry and were normalized to control (non-transfected) cells.

FIG. 8 is a graph showing reduction of CELF1 protein levels in a concentration-dependent manner after treatments with 0, 30, 60 and 180 μM of Compound II in Cl5-DM1 myotube sample. Data were collected by Western blot and analyzed by densitometry and were normalized to GADPH. Fold change is relative to control (0 μM of Compound II) and control is set to 1.0.

FIG. 9 is a graph showing reduction of CELF1 protein levels in a concentration-dependent manner after treatments with 0, 30, 60 and 180 μM of Compound II in DM1 patient myotube sample. Data were collected by Western blot and analyzed by densitometry and were normalized to GADPH. Fold change is relative to control (0 μM of Compound II) and control is set to 1.0.

FIG. 10 is a graph showing reduction of CELF1 protein levels in a concentration-dependent manner after treatments with 0, 30, 60 and 180 μM of Compound I in DM1 patient myotube sample. Data were collected by Western blot and analyzed by densitometry and were normalized to GADPH. Fold change is relative to control (0 μM of Compound I) and control is set to 1.0.

FIG. 11A is a graph showing the suppression of the ion channel ATP1B1 after 72 h treatment with 0, 30, 60 and 180 μM of Compound II in myotubes from one DM1 patient sample. Data were collected by Western blot and analyzed by densitometry and were normalized to GAPDH. Fold change is shown relative to control (0 μM of Compound II) and control value is set to 1.0. Error bar indicate mean±SD (n=3 independent experiments). *p<0.05, **p<0.01, ***p<0.001.

FIG. 11B is a graph showing the suppression of the ion channel SERCA2 after 72 h treatment with 0, 30, 60 and 180 μM of Compound II in myotubes from one DM1 patient sample. Data were collected by Western blot and analyzed by densitometry and were normalized to GAPDH. Fold change is shown relative to control (0 μM of Compound II) and control value is set to 1.0. Error bar indicate mean±SD (n=3 independent experiments). *p<0.05, **p<0.01, ***p<0.001.

FIG. 11C is a graph showing the suppression of the ion channel KCNJ2 after 72 h treatment with 0, 30, 60 and 180 μM of Compound II in myotubes from one DM1 patient sample. Data were collected by Western blot and analyzed by densitometry and were normalized to GAPDH. Fold change is shown relative to control (0 μM of Compound II) and control value is set to 1.0. Error bar indicate mean±SD (n=3 independent experiments). *p<0.05, **p<0.01, ***p<0.001.

FIG. 11D is a graph showing the suppression of the ion channel CACNA1S after 72 h treatment with 0, 30, 60 and 180 μM of Compound II in myotubes from one DM1 patient sample. Data were collected by Western blot and analyzed by densitometry and were normalized to GAPDH. Fold change is shown relative to control (0 μM of Compound II) and control value is set to 1.0. Error bar indicate mean±SD (n=3 independent experiments). *p<0.05, **p<0.01, ***p<0.001.

FIG. 11E is a graph showing the suppression of the ion channel SERCA1 after 72 h treatment with 0, 30, 60 and 180 μM of Compound II in myotubes from one DM1 patient sample. Data were collected by Western blot and analyzed by densitometry and were normalized to GAPDH. Fold change is shown relative to control (0 μM of Compound II) and control value is set to 1.0. Error bar indicate mean±SD (n=3 independent experiments). *p<0.05, **p<0.01, ***p<0.001.

FIG. 11F is a graph showing the suppression of the ion channel CACNA1C after 72 h treatment with 0, 30, 60 and 180 μM of Compound II in myotubes from one DM1 patient sample. Data were collected by Western blot and analyzed by densitometry and were normalized to GAPDH. Fold change is shown relative to control (0 μM of Compound II) and control value is set to 1.0. Error bar indicate mean±SD (n=3 independent experiments). *p<0.05, **p<0.01, ***p<0.001.

FIG. 12A is a graph showing the suppression of the ion channel ATP1B1 after 72 h treatment with 0, 30, 60 and 180 μM of Compound I in myotubes from one DM1 patient sample. Data were collected by Western blot and analyzed by densitometry and were normalized to GAPDH. Fold change is shown relative to control (0 μM of Compound I) and control value is set to 1.0. Error bar indicate mean±SD (n=3 independent experiments). *p<0.05, **p<0.01, ***p<0.001.

FIG. 12B is a graph showing the suppression of the ion channel SERCA2 after 72 h treatment with 0, 30, 60 and 180 μM of Compound I in myotubes from one DM1 patient sample. Data were collected by Western blot and analyzed by densitometry and were normalized to GAPDH. Fold change is shown relative to control (0 μM of Compound I) and control value is set to 1.0. Error bar indicate mean±SD (n=3 independent experiments). *p<0.05, **p<0.01, ***p<0.001.

FIG. 12C is a graph showing the suppression of the ion channel KCNJ2 after 72 h treatment with 0, 30, 60 and 180 μM of Compound I in myotubes from one DM1 patient sample. Data were collected by Western blot and analyzed by densitometry and were normalized to GAPDH. Fold change is shown relative to control (0 μM of Compound I) and control value is set to 1.0. Error bar indicate mean±SD (n=3 independent experiments). *p<0.05, **p<0.01, ***p<0.001.

FIG. 12D is a graph showing the suppression of the ion channel CACNA1S after 72 h treatment with 0, 30, 60 and 180 μM of Compound I in myotubes from one DM1 patient sample. Data were collected by Western blot and analyzed by densitometry and were normalized to GAPDH. Fold change is shown relative to control (0 μM of Compound I) and control value is set to 1.0. Error bar indicate mean±SD (n=3 independent experiments). *p<0.05, **p<0.01, ***p<0.001.

FIG. 12E is a graph showing the suppression of the ion channel SERCA1 after 72 h treatment with 0, 30, 60 and 180 μM of Compound I in myotubes from one DM1 patient sample. Data were collected by Western blot and analyzed by densitometry and were normalized to GAPDH. Fold change is shown relative to control (0 μM of Compound I) and control value is set to 1.0. Error bar indicate mean±SD (n=3 independent experiments). *p<0.05, **p<0.01, ***p<0.001.

FIG. 12F is a graph showing the suppression of the ion channel CACNA1C after 72 h treatment with 0, 30, 60 and 180 μM of Compound I in myotubes from one DM1 patient sample. Data were collected by Western blot and analyzed by densitometry and were normalized to GAPDH. Fold change is shown relative to control (0 μM of Compound I) and control value is set to 1.0. Error bar indicate mean±SD (n=3 independent experiments). *p<0.05, **p<0.01, ***p<0.001.

FIG. 13A is a graph showing the suppression of the ion channel ATP1B1 after 72 h treatment with 0, 30, 60 and 180 μM of Compound II in myotubes from one DM2 patient sample. Data were collected by Western blot and analyzed by densitometry and were normalized to GAPDH. Fold change is shown relative to control (0 μM of Compound II) and control value is set to 1.0. Error bar indicate mean±SD (n=3 independent experiments). *p<0.05, **p<0.01, ***p<0.001.

FIG. 13B is a graph showing the suppression of the ion channel KCNJ2 after 72 h treatment with 0, 30, 60 and 180 μM of Compound II in myotubes from one DM2 patient sample. Data were collected by Western blot and analyzed by densitometry and were normalized to GAPDH. Fold change is shown relative to control (0 μM of Compound II) and control value is set to 1.0. Error bar indicate mean±SD (n=3 independent experiments). *p<0.05, **p<0.01, ***p<0.001.

FIG. 13C is a graph showing the suppression of the ion channel CACNA1S after 72 h treatment with 0, 30, 60 and 180 μM of Compound II in myotubes from one DM2 patient sample. Data were collected by Western blot and analyzed by densitometry and were normalized to GAPDH. Fold change is shown relative to control (0 μM of Compound II) and control value is set to 1.0. Error bar indicate mean±SD (n=3 independent experiments). *p<0.05, **p<0.01, ***p<0.001.

FIG. 14A is a graph showing the suppression of the ion channel ATP1B1 after 72 h treatment with 0, 30, 60 and 180 μM of Compound II in myotubes from the DM1-Cl5 cell line. Data were collected by Western blot and analyzed by densitometry and were normalized to GAPDH. Fold change is shown relative to control (0 μM of Compound II) and control value is set to 1.0. Error bar indicate mean±SD (n=3 independent experiments). *p<0.05, **p<0.01, ***p<0.001.

FIG. 14B is a graph showing the suppression of the ion channel SERCA2 after 72 h treatment with 0, 30, 60 and 180 μM of Compound II in myotubes from the DM1-Cl5 cell line. Data were collected by Western blot and analyzed by densitometry and were normalized to GAPDH. Fold change is shown relative to control (0 μM of Compound II) and control value is set to 1.0. Error bar indicate mean±SD (n=3 independent experiments). *p<0.05, **p<0.01, ***p<0.001.

FIG. 14C is a graph showing the suppression of the ion channel KCNJ2 after 72 h treatment with 0, 30, 60 and 180 μM of Compound II in myotubes from the DM1-Cl5 cell line. Data were collected by Western blot and analyzed by densitometry and were normalized to GAPDH. Fold change is shown relative to control (0 μM of Compound II) and control value is set to 1.0. Error bar indicate mean±SD (n=3 independent experiments). *p<0.05, **p<0.01, ***p<0.001.

FIG. 14D is a graph showing the suppression of the ion channel CACNA1S after 72 h treatment with 0, 30, 60 and 180 μM of Compound II in myotubes from the DM1-Cl5 cell line. Data were collected by Western blot and analyzed by densitometry and were normalized to GAPDH. Fold change is shown relative to control (0 μM of Compound II) and control value is set to 1.0. Error bar indicate mean±SD (n=3 independent experiments). *p<0.05, **p<0.01, ***p<0.001.

FIG. 14E is a graph showing the suppression of the ion channel SERCA1 after 72 h treatment with 0, 30, 60 and 180 μM of Compound II in myotubes from the DM1-Cl5 cell line. Data were collected by Western blot and analyzed by densitometry and were normalized to GAPDH. Fold change is shown relative to control (0 μM of Compound II) and control value is set to 1.0. Error bar indicate mean±SD (n=3 independent experiments). *p<0.05, **p<0.01, ***p<0.001.

FIG. 15 is a model showing CUG^{exp} binds Lin28 causing loss of miRNA-mediated control of ion channels linked to clinical hallmarks of myotonic dystrophy.

FIG. 16A is a graph showing increase in CLCN1 levels after 72 h treatment with 0, 30, 60 and 180 μM of Compound II in primary skeletal myotubes from one DM1 patient. Data were collected by Western blot and analysed by densitometry and were normalized to GADPH. Fold change relative to control (0 μM of Compound II) and control value is set to 1.0. Error bar indicates mean±SD (n=3 independent experiments). *p<0.05, **p<0.01, ***p<0.001.

FIG. 16B is a graph showing increase in CLCN1 levels after 72 h treatment with 0, 30, 60 and 180 μM of Compound II in primary skeletal myotubes from cl5-DM1 cell line. Data were collected by Western blot and analysed by densitometry and were normalized to GADPH. Fold change relative to control (0 μM of Compound II) and control value is set to 1.0. Error bar indicates mean±SD (n=2 independent experiments). *p<0.05, **p<0.01, ***p<0.001.

FIG. 17A is a graph showing reduced expression of PKC levels after 72 h treatment with 0, 30, 60 and 180 μM of Compound II in primary skeletal myotubes from one DM1 patient. Data were collected by Western blot and analysed by densitometry and were normalized to GADPH. Fold change relative to control (0 μM of Compound II) and control value is set to 1.0. Error bar indicates mean±SD (n=3 independent experiments). *p<0.05, **p<0.01, ***p<0.001.

FIG. 17B is a graph showing reduced expression of PKC levels after 72 h treatment with 0, 30, 60 and 180 μM of Compound II in primary skeletal myotubes from cl5-DM1 cell line. Data were collected by Western blot and analysed by densitometry and were normalized to GADPH. Fold change relative to control (0 μM of Compound II) and control value is set to 1.0. Error bar indicates mean±SD (n=3 independent experiments). *p<0.05, **p<0.01, ***p<0.001.

FIG. 17C is a graph showing reduced expression of PKC levels after 72 h treatment with 0, 30, 60 and 180 μM of Compound I in primary skeletal myotubes from one DM1 patient. Data were collected by Western blot and analysed by densitometry and were normalized to GADPH. Fold change relative to control (0 μM of Compound I) and control value is set to 1.0. Error bar indicates mean±SD (n=3 independent experiments). *p<0.05, **p<0.01, ***p<0.001.

FIG. 17D is a graph showing reduced expression of phosphorylated PKC levels after 72 h treatment with 0, 30, 60 and 180 μM of Compound I in primary skeletal myotubes from one DM1 patient. Data were collected by Western blot and analysed by densitometry and were normalized to GADPH. Fold change relative to control (0 μM of Compound I) and control value is set to 1.0. Error bar indicates mean±SD (n=3 independent experiments). *p<0.05, **p<0.01, ***p<0.001.

### DETAILED DESCRIPTION OF THE INVENTION

Unless defined otherwise, all technical and scientific terms used herein have the same meanings as commonly understood by one of ordinary skill in the art to which this invention belongs.

### Definitions

As used herein “a” or “an” means one or more, unless specifically indicated to mean only one.

The term “about” where used means especially ±10%, ±5% or ±3% (referring to the given numeric value, respectively), if not indicated otherwise. In each of the invention embodiments, “about” can be deleted.

“Administration” as used herein encompasses all suitable means of providing a substance to a subject. Common routes include oral, sublingual, transmucosal, transdermal, rectal, vaginal, subcutaneous, intramuscular, intravenous, intra-arterial, intrathecal, via catheter, via implant etc.

“Subject” as used herein includes any vertebrate animal, including equine, ovine, caprine, bovine, porcine, avian, canine, feline and primate species. Preferably a “subject” as used herein is a human.

As used herein, the term “effective amount” refers to an amount necessary or sufficient to realize a desired effect. Preferably, the term “effective amount” refers to an amount of Compound I and/or Compound II of the present invention that (i) treats or prevents the particular disease, medical condition, or disorder (e.g., DM1 or DM2), (ii) attenuates, ameliorates, or eliminates one or more symptoms of the particular disease, medical condition, or disorder (e.g., DM1 or DM2), or (iii) prevents or delays the onset of one or more symptoms of the particular disease, medical condition, or disorder (e.g., DM1 or DM2) described herein. In preferred embodiments, therapeutically effective amounts of the inventive compounds are administered under the guidance of a physician, and pharmaceutical compositions thereof will contain the inventive compounds in conjunction with a conventional, pharmaceutically or veterinary acceptable carrier. A therapeutically effective amount is considered to be a predetermined amount calculated to achieve the desired effect. The required dosage will vary with the particular treatment and with the duration of desired treatment.

The term “preventing” as used herein is understood to mean delaying the appearance of clinical symptoms of the state, disorder or condition developing in a subject that may be afflicted with or predisposed to the state, disorder or condition but does not yet experience or display clinical or subclinical symptoms of the state, disorder or condition.

The term “treating” as used herein is understood to mean inhibiting the state, disorder or condition (e.g. arresting, reducing or delaying the development of the disease, or a relapse thereof in case of maintenance treatment, of at least one clinical or subclinical symptom thereof); and/or relieving the condition (i.e. causing regression of the state, disorder or condition or at least one of its clinical or subclinical symptoms). Preferably the state, disorder or condition to be prevented or treated is DM, more preferably DM1 and/or DM2.

The terms “pharmaceutically acceptable” or “therapeutically acceptable” refers to a substance which does not interfere with the effectiveness or the biological activity of the active ingredients and which is not toxic to the host. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, hydroiodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. Preferred pharmaceutically acceptable salts include hydrochloride, hydrobromide, sulphate, phosphate, tannate, oxalate, fumarate, gluconate, alginate, maleate, acetate, citrate, benzoate, succinate, malate, ascorbate, tartrate and the like.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied.

A “solvate” refers to an association or complex of one or more solvent molecules and a compound of the present invention. Examples of solvents that form solvates include, but are not limited to, water, isopropanol, ethanol, methanol, dimethyl sulfoxide (DMSO), ethyl acetate, acetic acid, and ethanolamine. The term “hydrate” refers to the complex where the solvent molecule is water.

The term “tautomers” refers to a set of compounds that have the same number and type of atoms, but differ in bond connectivity and are in equilibrium with one another. A “tautomer” is a single member of this set of compounds. Typically a single tautomer is drawn but it is understood that this single structure is meant to represent all possible tautomers that might exist. Examples include enol-ketone tautomerism. When a ketone is drawn it is understood that both the enol and ketone forms are part of the present disclosure.

The term “stereoisomers” refers to compounds, which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

The terms “reducing” and “inhibiting” have their commonly understood meaning of lessening or decreasing.

As used herein, the term “for use” as used in, for example, “Compound I and/or Compound II for use in treatment or prevention of a disease” shall disclose also (i) the corresponding method of treatment or prevention of said disease in a subject in need thereof; and (ii) the corresponding use of Compound I and/or Compound II in the manufacture of a medicament for the treatment or prevention of said disease.

As used herein, the term “muscle atrophy” is understood to mean the wasting (including progressive wasting) or thinning of muscle mass in a subject. “Myofiber atrophy” is understood to be the wasting or thinning of myofibers.

As used herein, the term “muscle myotonia” is understood to mean a neuromuscular condition in which the relaxation of muscle is impaired.

As used herein, the term “fibrosis” is understood to mean a thickening or scarring of tissue, preferably muscle tissue.

The term “Lin28-miRNA” is understood to mean a miRNA, the expression and/or maturation of which is affected by, preferably controlled by, the activity of Lin28. In preferred embodiments, a Lin-28-miRNA is a miRNA, the expression of which is reduced and/or eliminated by the activity of Lin28.

As used herein, the term “DM-related miRNA” or “DM-miRNA” is understood to mean a miRNA, the expression of which is increased or decreased in cells of a DM patient compared to cells of a healthy volunteer. Preferably, levels of a “DM-related miRNA” or “DM-miRNA” is decreased the cells of a DM patient compared to cells of a healthy volunteer.

### Compounds of the Invention

In one aspect, the present invention provides a compound, wherein said compound is Compound I and/or Compound II, or a pharmaceutically acceptable salt, stereoisomer, enantiomer, tautomer, hydrate or solvate thereof, for use in a method of preventing or treating a myotonic dystrophy in a subject:

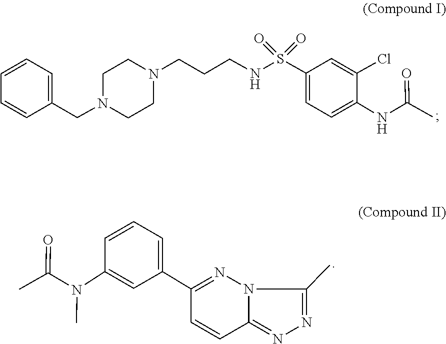

In some embodiments, the compound is Compound I, or a pharmaceutically acceptable salt, stereoisomer, enantiomer, tautomer, hydrate or solvate thereof.

In some embodiments, the compound is Compound II, or a pharmaceutically acceptable salt, stereoisomer, enantiomer, tautomer, hydrate or solvate thereof.

In one aspect, the present invention provides a pharmaceutical composition comprising Compound I and/or Compound II, and a pharmaceutically acceptable carrier or excipient.

In one aspect, the present invention provides a pharmaceutical composition comprising Compound I, and a pharmaceutically acceptable carrier or excipient.

In one aspect, the present invention provides a pharmaceutical composition comprising Compound II, and a pharmaceutically acceptable carrier or excipient.

### Use of the Inventive Compounds

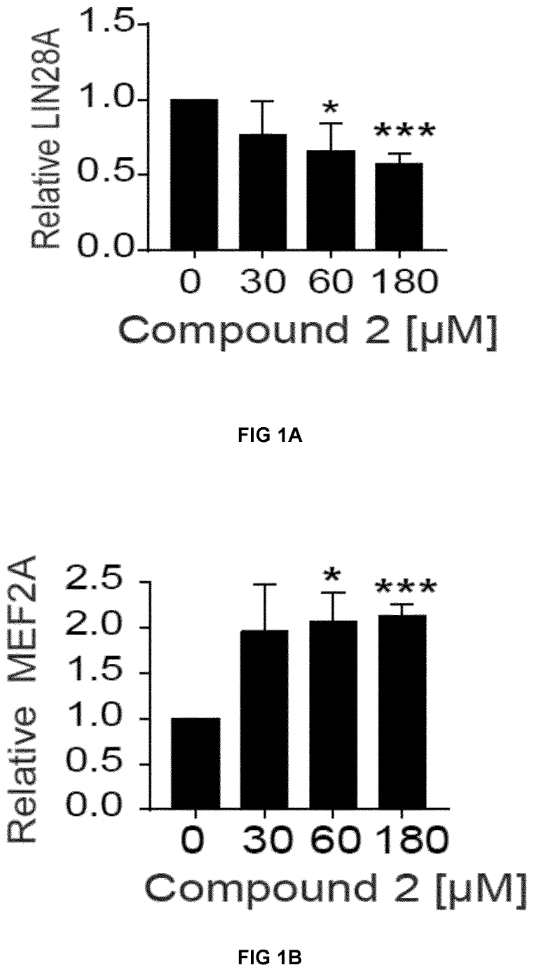

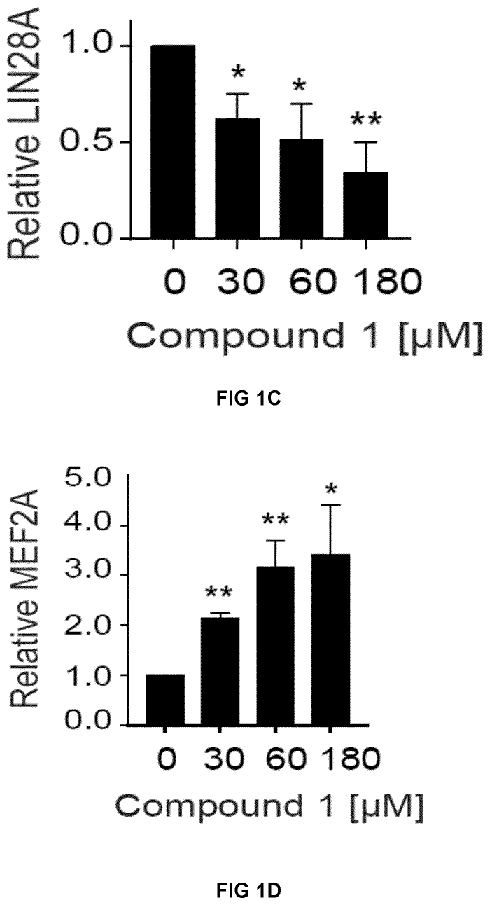

As described in Examples 1 and in FIGS. 1A-1D, 2A-2B, 3A-3B, 7, 8, 9, 10, 11A-11F, 12A-12F, 13A-13C, and 14A-14E, treatment of cells derived from DM patients with Compound I and/or Compound II can increase the expression of proteins that are downregulated in DM, and can decrease the expression of proteins that are upregulated in DM.

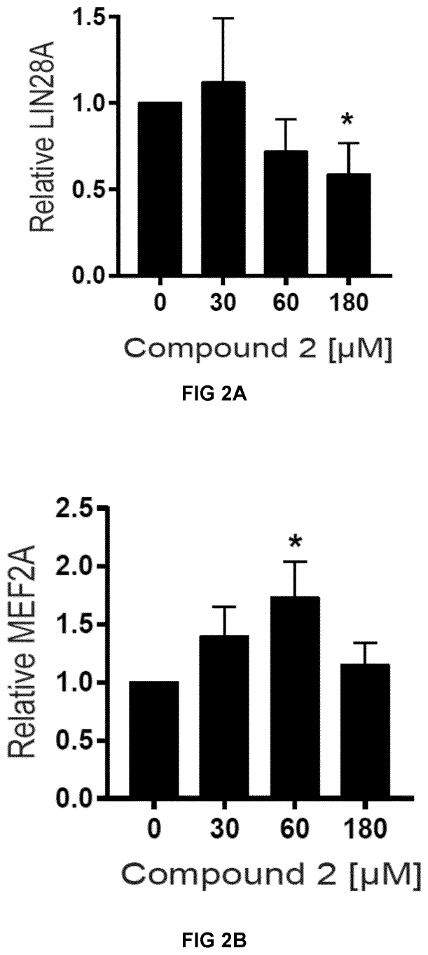

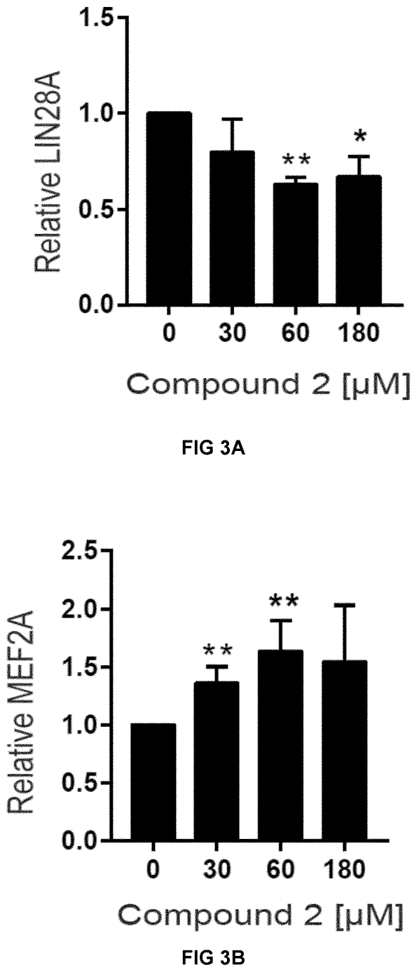

As shown in FIGS. 1A-1D, treatment of primary skeletal myotubes from a DM1 patient with Compound I and Compound II led to a decrease in Lin28A protein concentration and an increase in MEF2A protein concentration in DM cells. FIG. 1A shows that Lin28A protein levels were reduced by 40-50% at 60 μM and 180 μM concentrations of Compound II in primary myotubes derived from a DM1 patient. A reduction in Lin28A protein level was also observed in immortalized myoblast cells (DM1-Cl5) as shown in FIG. 3A. A similar but slightly weaker reduction in Lin28A protein levels was seen in primary myotubes derived from a DM2 patient, as shown in FIG. 2A.

MEF2A protein was also increased in primary myotubes derived from a DM1 patient, a DM2 patient, and immortalized myoblast cells (DM1-Cl5) in response to treatment with Compound II, as shown in FIGS. 1B, 2B, and 3B, respectively. Essentially identical observations were made for the use of Compound I, as shown in FIGS. 1C and 1D.

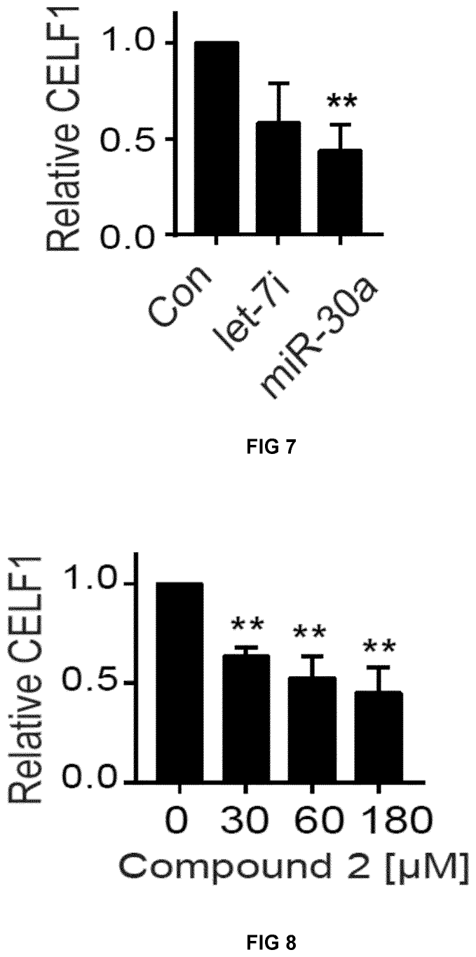

FIG. 7 is a graph showing a reduction in CELF1 expression after transfection of let-7i and miR-30a mimics in Ctrol-C148 cell lines compared to mock transfection.

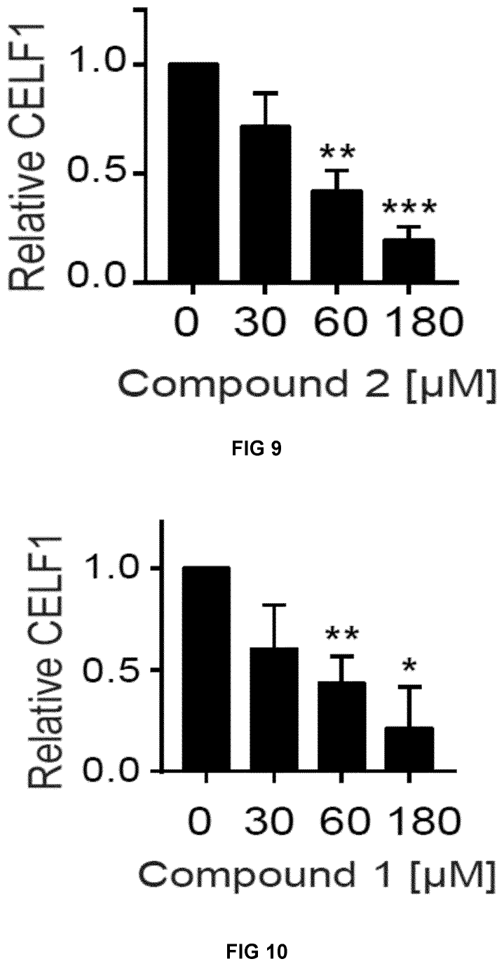

FIGS. 8-9 demonstrate that Compound II led to a reduction of CELF1 levels in DM1 cells by more than 50% (FIG. 8, Cl5-DM1 cells; FIG. 9, DM1 patient myotube sample). Similarly, FIG. 10 demonstrates that Compound I led to a reduction of CELF1 levels of more than 50% in a DM1 patient myotube sample. Without wishing to be bound by theory, the inhibition of CELF1 by compounds of the invention suggests that the inventive compounds can alleviate muscle wasting in a subject in need thereof, e.g., in a subject with DM.

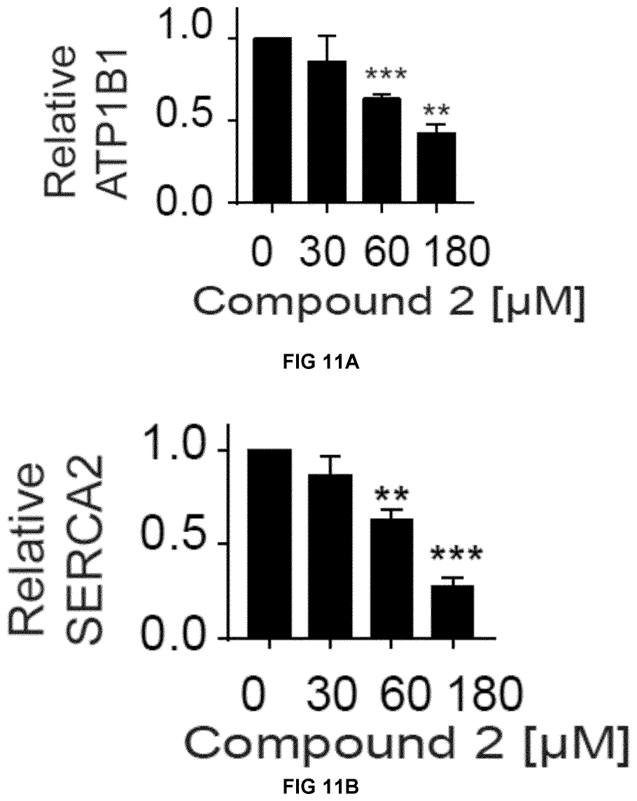

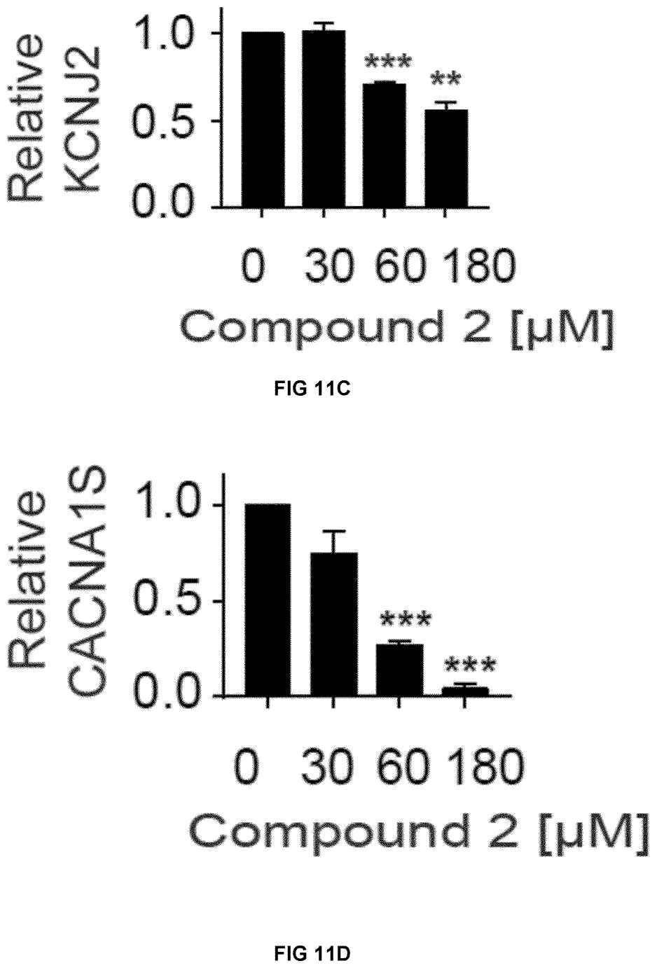

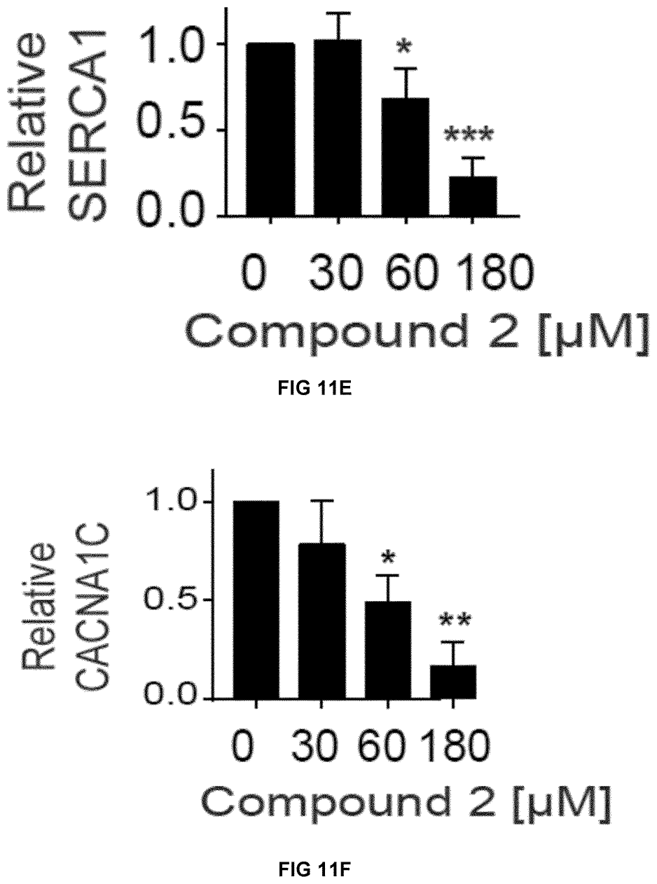

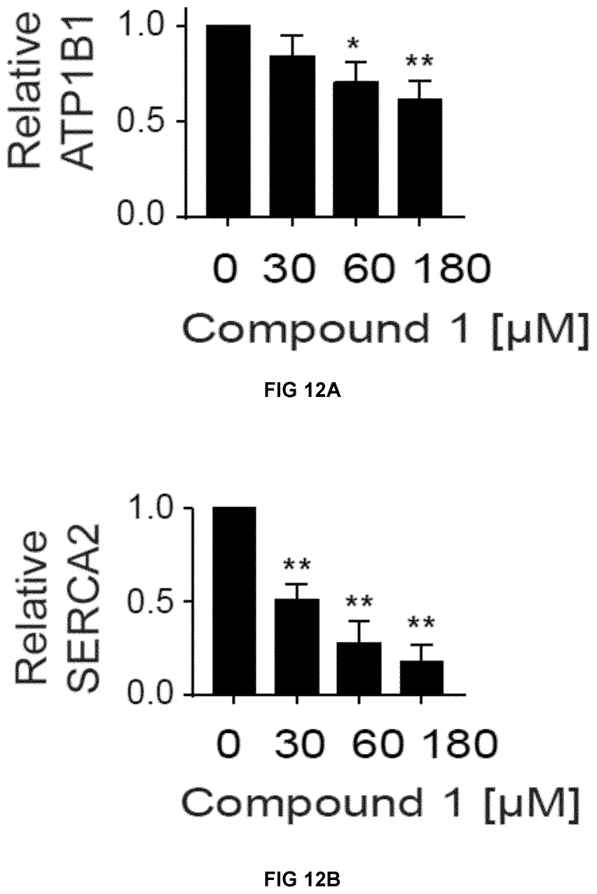

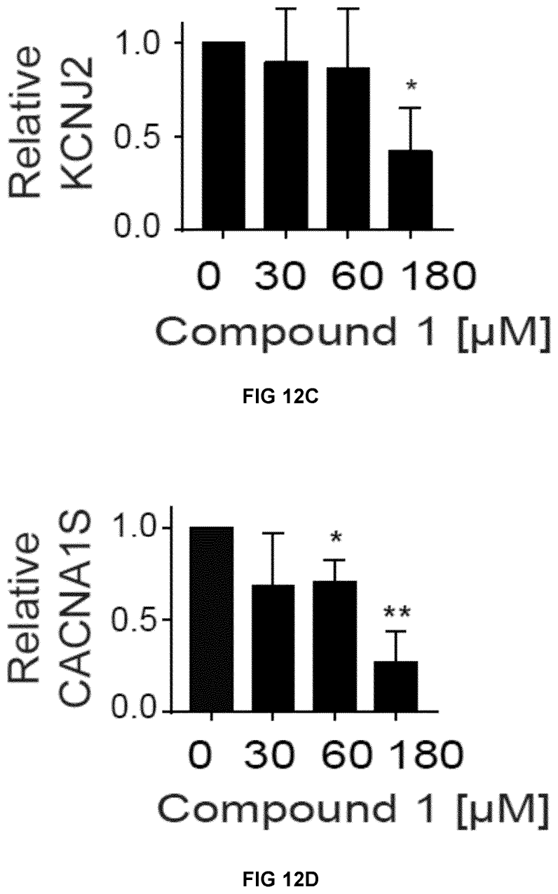

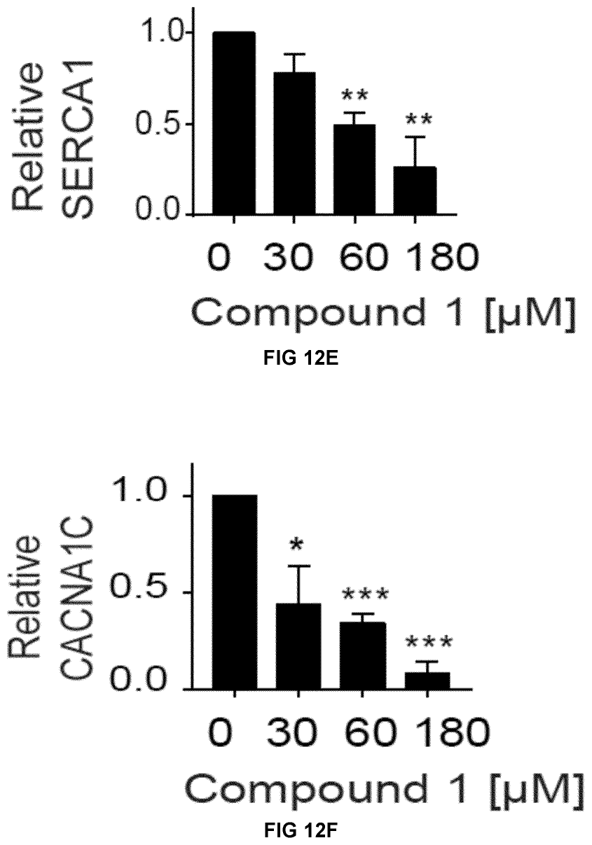

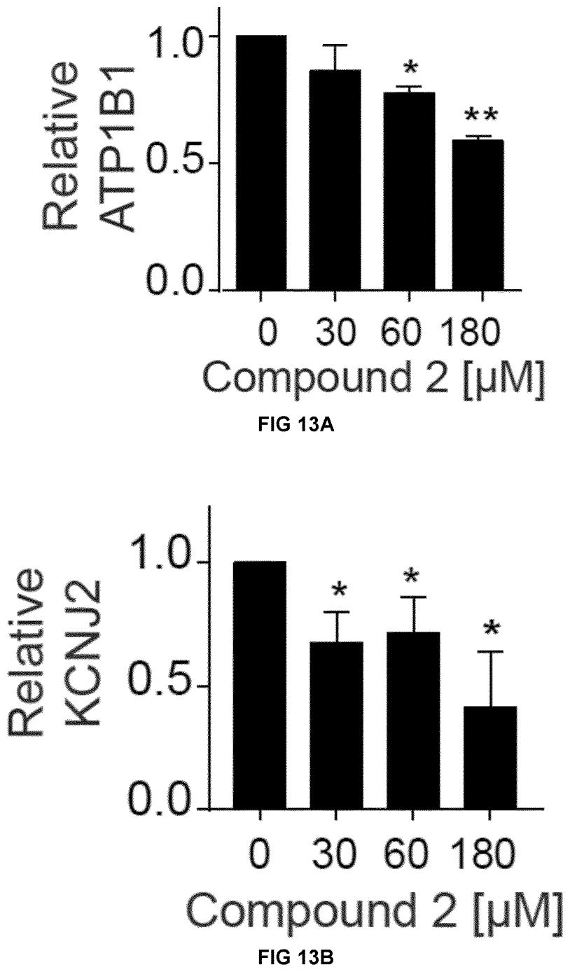

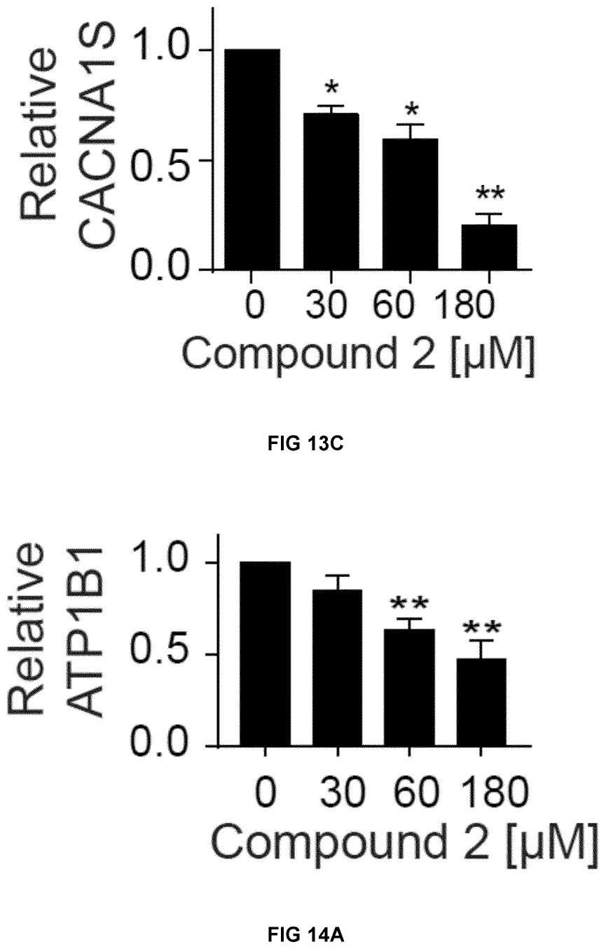

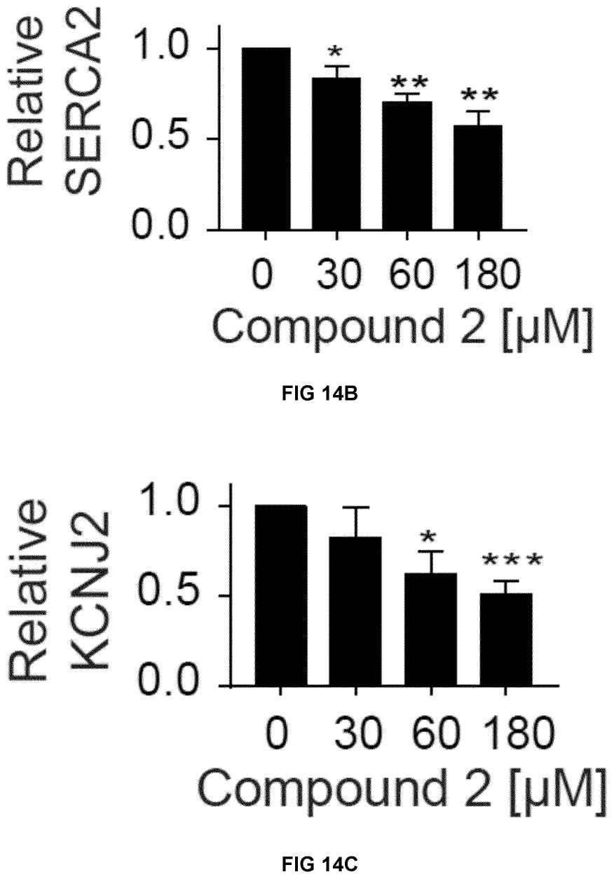

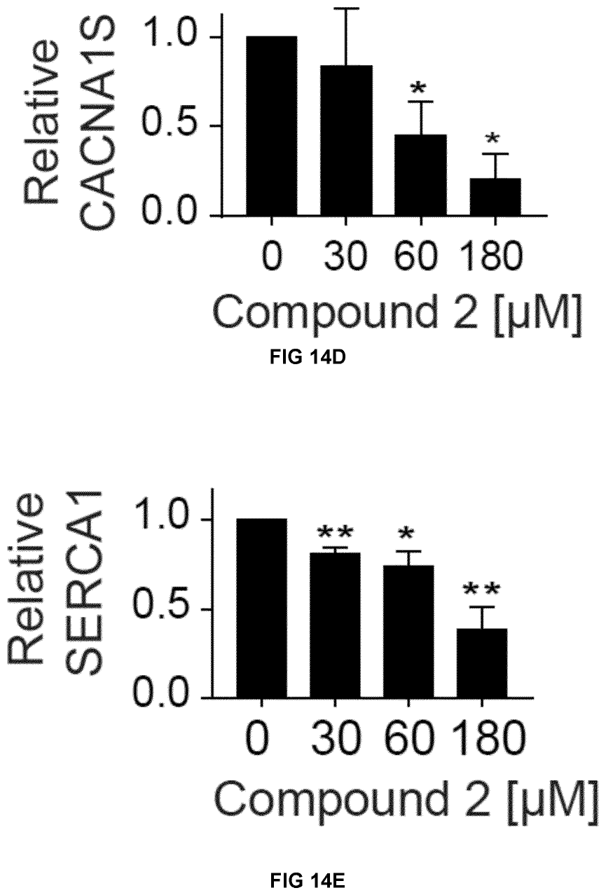

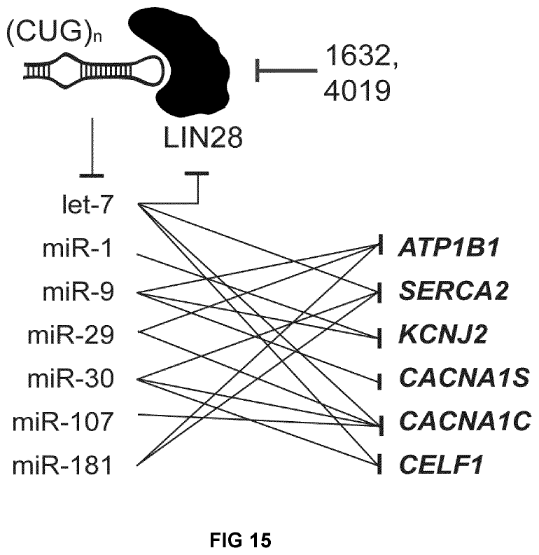

FIGS. 11A-11F, 12A-12F, 13A-13C, and 14A-14E demonstrate that treatment of DM cells with the inventive compounds at graded concentrations reduced abundance of ion channels associated with myotonia, muscle wasting, and cardiac conduction, e.g., by 30-80%. FIGS. 11A-11F demonstrate that treatment of myotubes from a DM1 patient sample with Compound II reduced the abundance of the ion channels ATP1B1, SERCA2, KCNJ2, CACNA1S, SERCA1, and CACNA1C. FIGS. 12A-12F demonstrate that treatment of myotubes from a DM1 patient sample with Compound I reduced the abundance of the ion channels ATP1B1, SERCA2, KCNJ2, CACNA1S, SERCA1, and CACNA1C from about 40% to about 95%. FIGS. 13A-C demonstrate that treatment of myotubes from a DM2 patient sample with Compound II reduced the abundance of the ion channels ATP1B1, KCNJ2, and CACNA1S from about 45% to about 85%. FIGS. 14A-E demonstrate that treatment of myotubes from the DM1-Cl5 cell line with Compound II reduced the abundance of the ion channels ATP1B1, SERCA2, KCNJ2, CACNA1S, and SERCA1 from about 50% to about 95%. Inhibition of SERCA1 and CACNA1C using Compound I was detectable at the lowest concentrations tested (30 μM), and expression of CACNA1S was almost abolished at 180 μM. Without wishing to be bound by theory, the results above suggest that treatment with the inventive compounds can be used to attenuate the muscle wasting and myotonia processes in a subject in need thereof, e.g., a subject with DM.

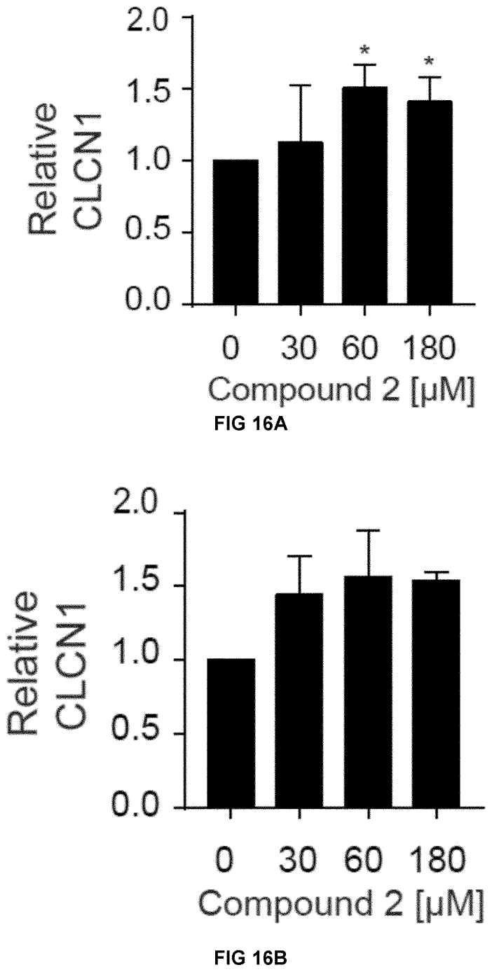

FIGS. 16A and 16B demonstrate that treatment of DM cells with the inventive compounds can increase levels of the ion channel CLCN1 in a concentration-dependent manner. FIG. 16A shows that treatment of cells derived from a DM1 patient caused a concentration-dependent increase in expression of CLCN1 after 72 h of treatment with Compound II. FIG. 16B shows a similar increase in cl5-DMT cells upon treatment with Compound II. As shown in FIGS. 16A and 16B, CLCN1 levels were increased to 150% relative to control cells in cells treated with the inventive compounds.

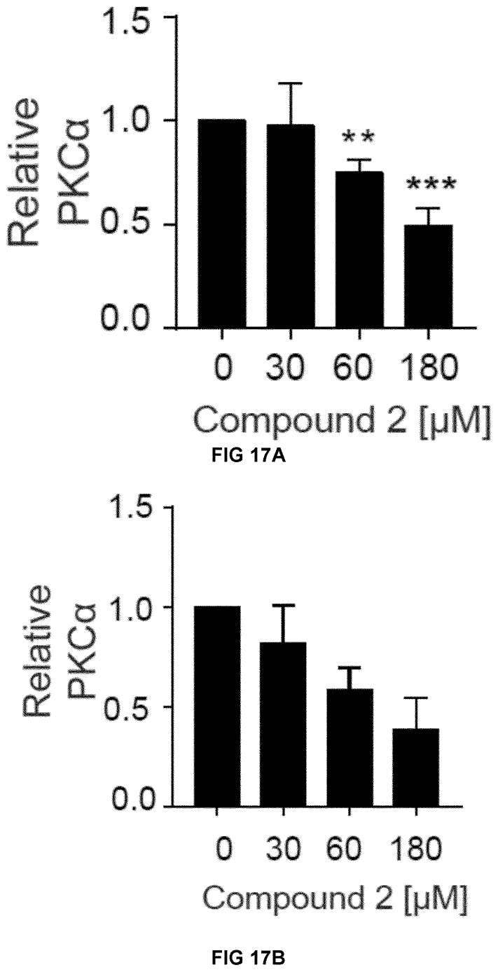

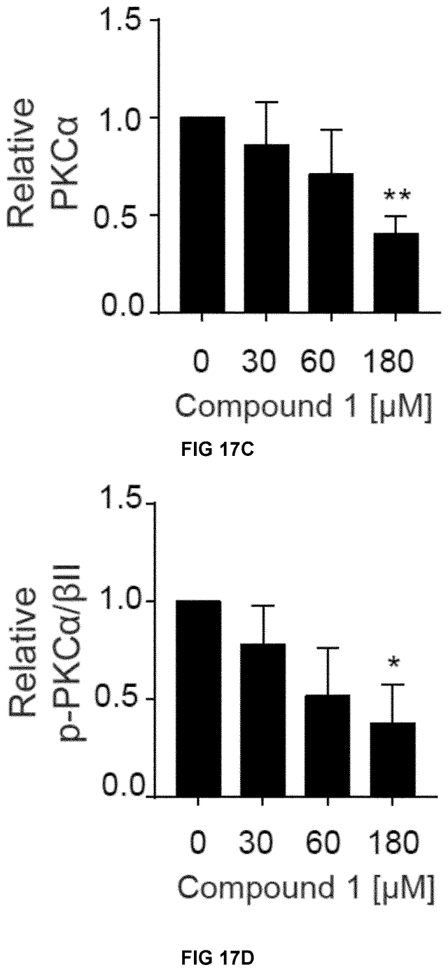

FIGS. 17A-17D demonstrate that treatment of DM cells with the inventive compounds reduced the expression of PKC and phosphorylated PKC (p-PKC) in a concentration-dependent manner. FIGS. 17A and 17B show reduced expression of PKCα in cells derived from a DM1 patient, and cl5-DM1 cells, respectively, as a function of concentration of Compound II. FIGS. 17C and 17D show reduced expression of PKCα (FIG. 17C) and phosphorylated PKCα (p-PKCα; FIG. 17D) in myotubes derived from a DM1 patient as a function of concentration. As shown in FIGS. 17A-17D, PKC and p-PKC was reduced by up to 50% relative to control cells in DM cells treated with the inventive compounds.

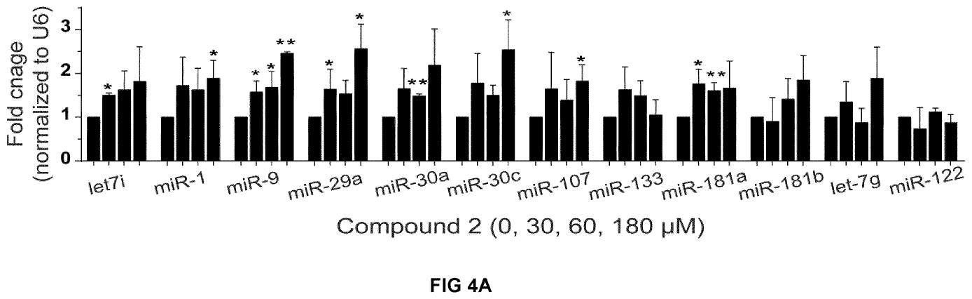

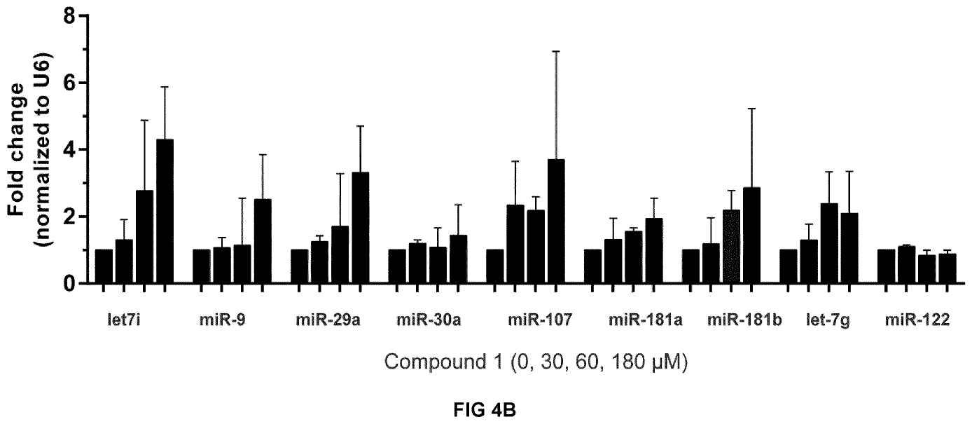

FIGS. 4A-4B demonstrate that treatment of DM cells with the inventive compounds de-repressed mature Lin28-miRNAs. As described in Example 2, myotubes treated with Compound I and/or Compound II were assayed for levels of Lin28-miRNAs by TaqMan-qPCR. As shown in FIGS. 4A and 4B, miR-122, a miRNA that is not bound nor regulated by Lin28, was unaffected by treatment with Compound I or Compound II. In contrast, let-7i and let-7g were increased up to 1.8-fold. The nine other miRNAs assayed (i.e., miR-1; miR-9; miR-29a; miR-30; miR-30c; miR-107; miR-133; miR-181a; and miR-181b) were similarly increased, with the maximum effect of Compound II observed for miR-30, which represses SERCA2 and MBNL1. miR-133 and miR-1, which are regulated by MEF2A, were similarly increased. Without wishing to be bound by theory, the results shown in FIGS. 4A-4B suggest that targeting Lin28 (e.g., by Compound I and/or Compound II) raised the levels of eleven DM-related miRNAs, with potential effects on their targetomes and the affected ion channels.

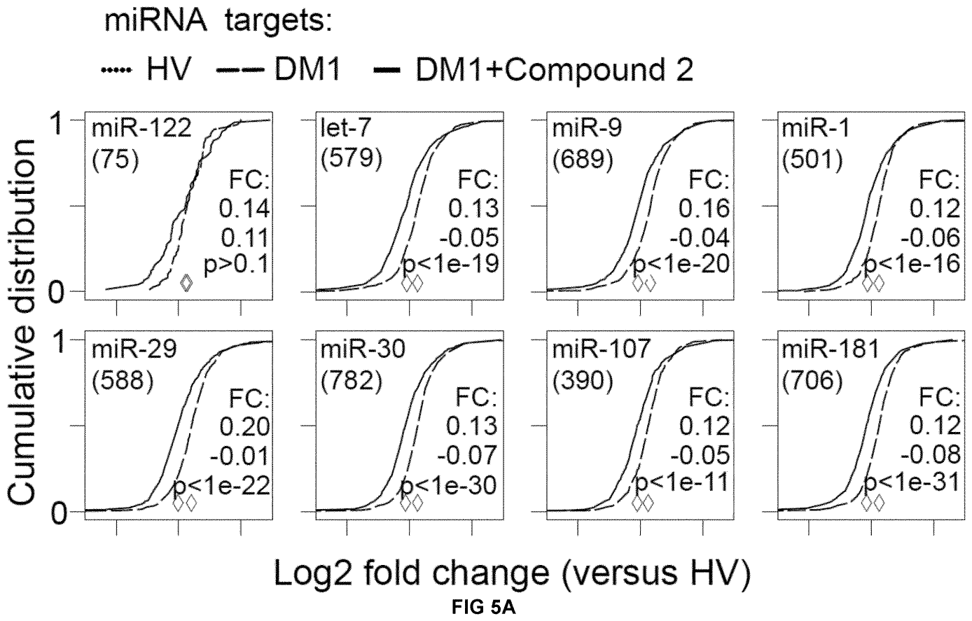

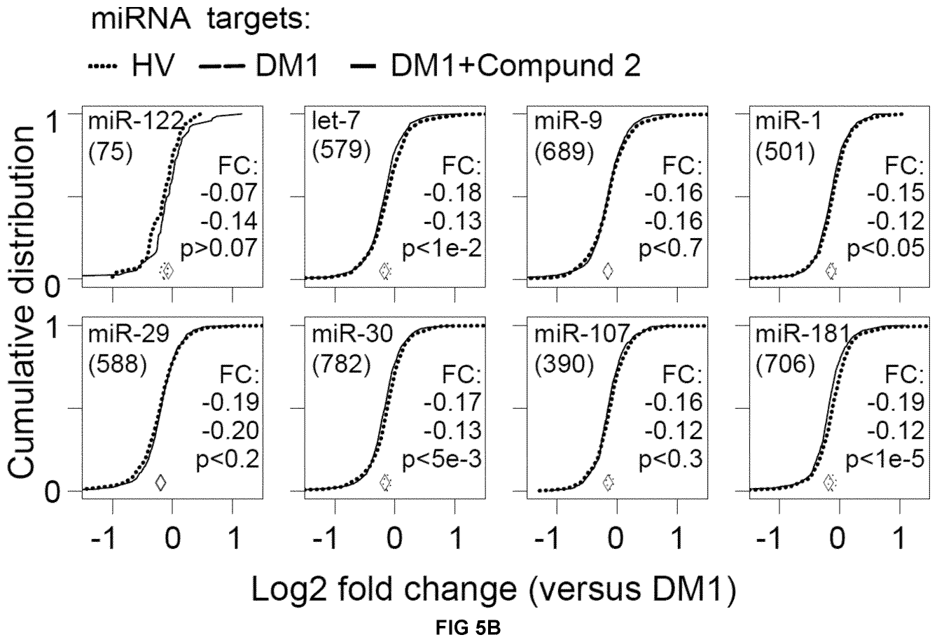

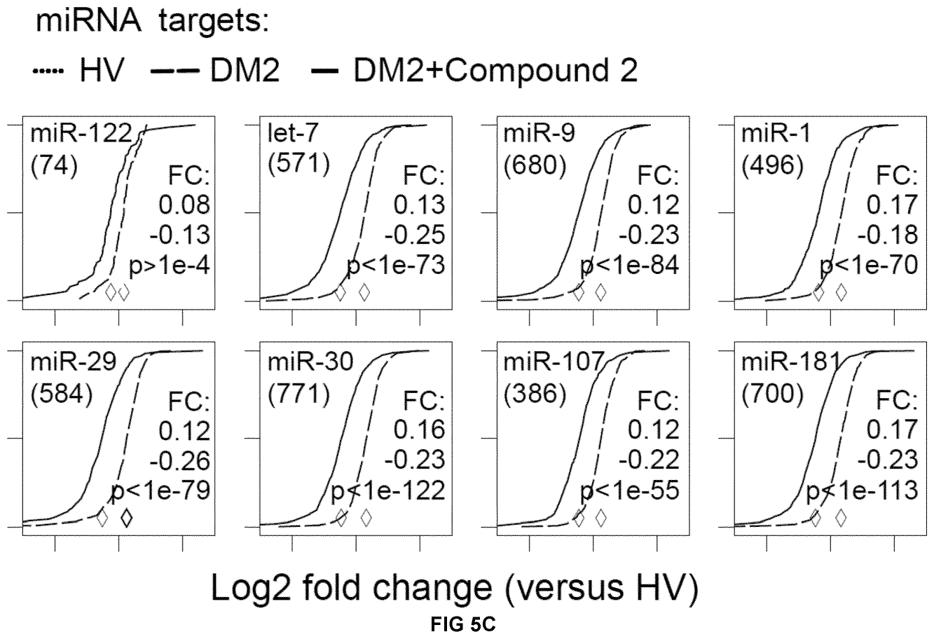

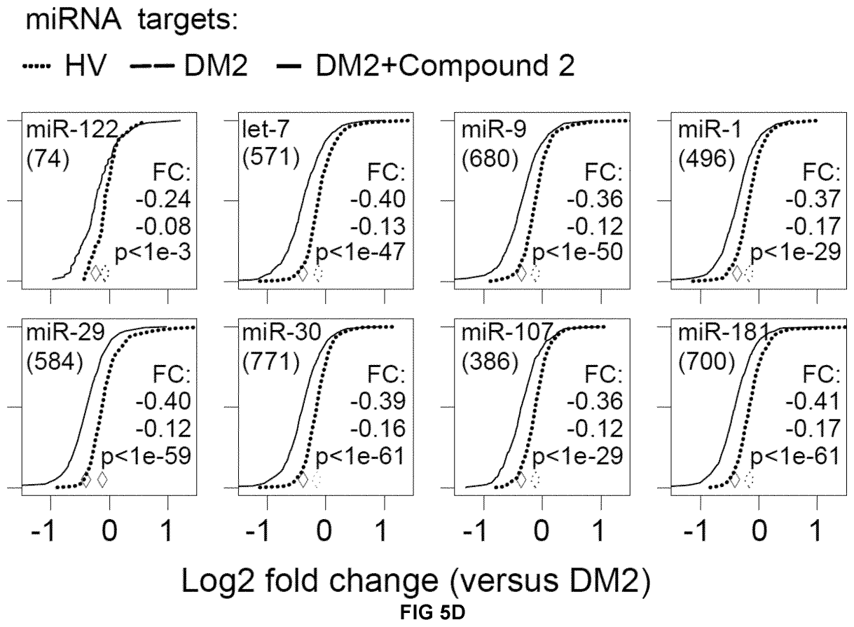

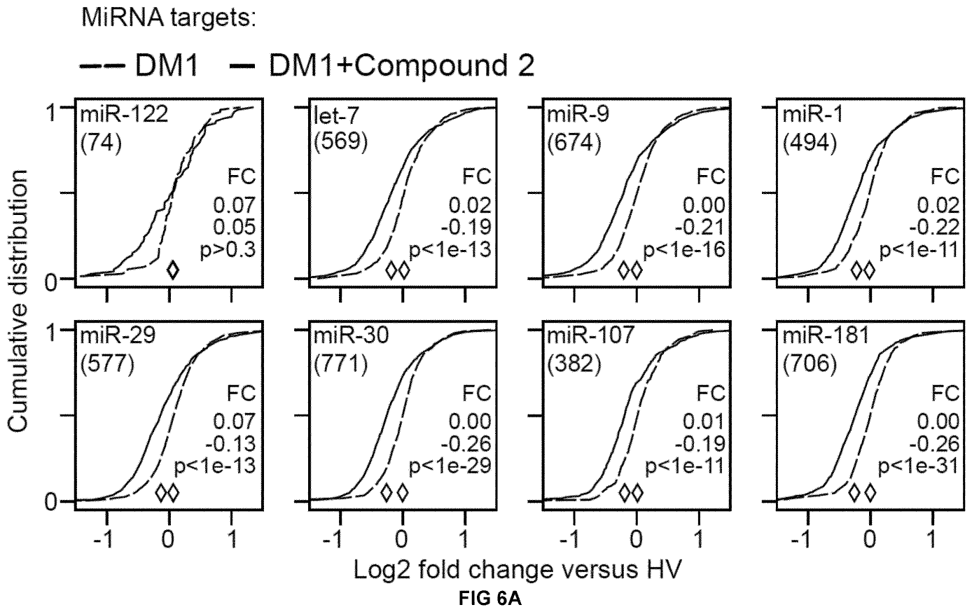

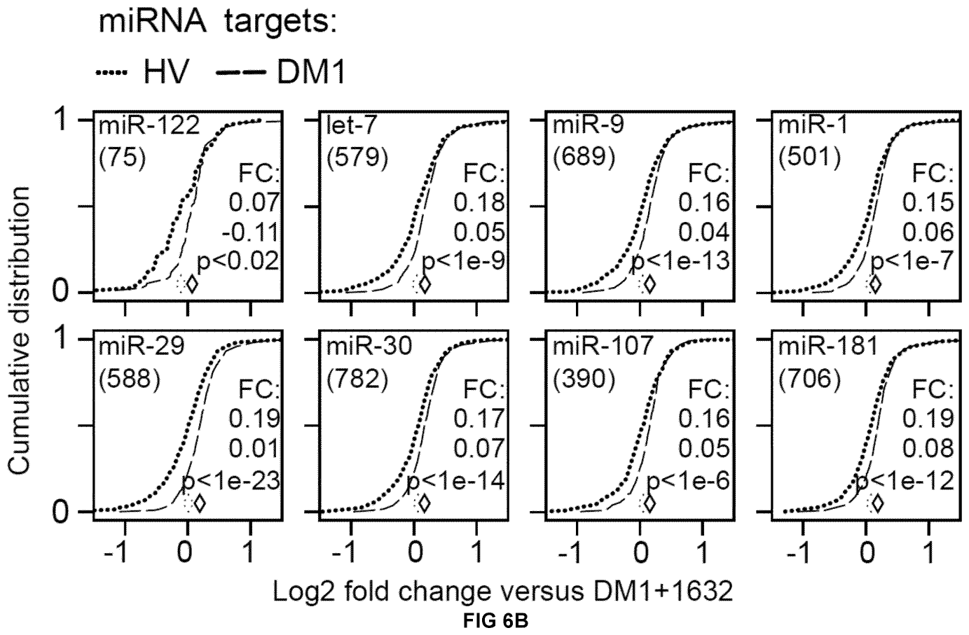

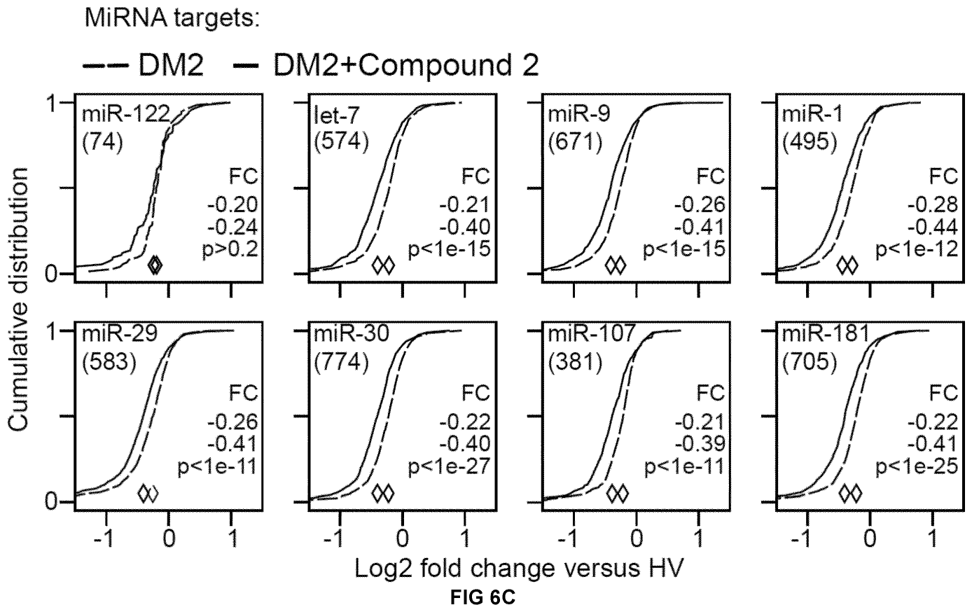

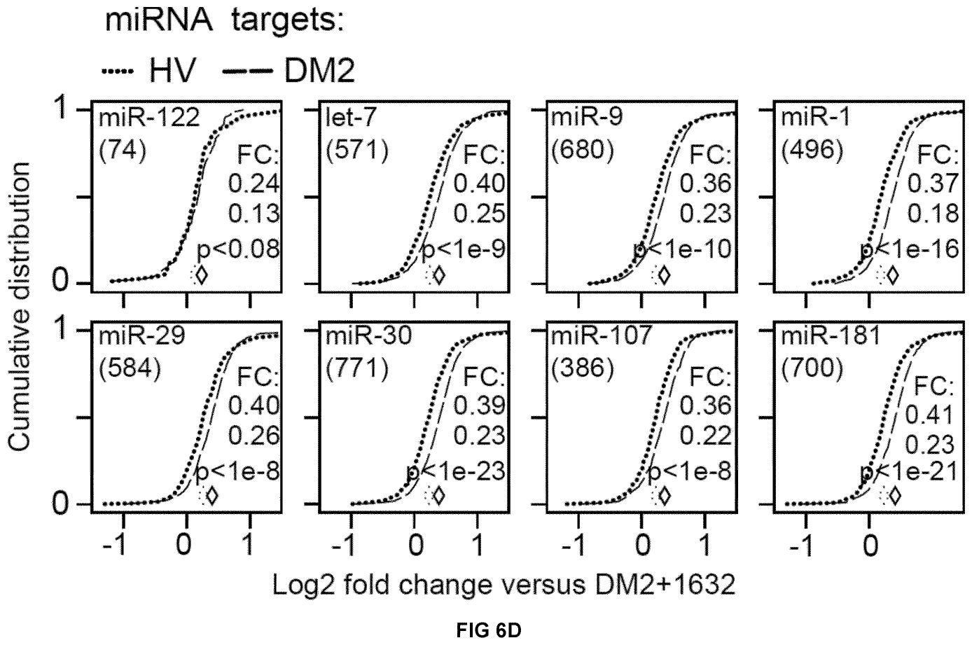

FIGS. 5A-5D and 6A-6D demonstrate the effect of treatment with Compound II on the targetomes of myoblasts and myotubes from DM patients and healthy volunteers as described in Example 3. As described in Example 3, total RNA was isolated from DM-patient and healthy cells, and cDNA libraries were prepared for three conditions (healthy volunteer (HV); DM; DM+Compound II) for DM1 and DM2 backgrounds. The expression levels of predicted targetomes of miRNAs let-7, miR-1, miR-9, miR-29, miR-30, miR-107 and miR-181 were then compared, using miR-122 as well as more than 2,000 transcripts that are not predicted targets of any miRNA as negative controls. Three pair-wise comparisons were performed to measure: i) how a targetome varies between healthy and diseased cells (HV versus DM), ii) how a targetome responds to treatment with the inventive compounds (DM versus DM+Compound II) and, iii) how a Compound II-treated DM-targetome compares to that of a healthy signature. For each pair-wise comparison, the third dataset was used as a reference and the fold changes were calculated for each target transcript (e.g. DM1 vs. DM1+Compound II, with fold changes calculated relative to HV).

Without wishing to be bound by theory, the strongest effects of a miRNA against a given target are expected at the protein level, where target repression by up to several fold can sometimes be observed (Bartel, D. P. (2018). Metazoan MicroRNAs. Cell 173, 20-51). This contrasts with the magnitude of the fold-change typically observed on the target mRNA levels, which can be substantial, but is usually weak (Agarwal et al., (2015). Predicting effective microRNA target sites in mammalian mRNAs. Elife 4; Imig et al., (2015). miR-CLIP capture of a miRNA targetome uncovers a lincRNA H19-miR-106a interaction. Nat Chem Biol 11, 107-114). This means that in some embodiments, the overall effect of a miRNA on its predicted mRNA targetome is rather small (Bartel, D. P. (2018). Metazoan MicroRNAs. Cell 173, 20-51). For instance, transfection of a selected miRNA into cells, which may increase its intracellular concentration more than 100-fold, may suppress its 500-member mRNA targetome by approximately 28%, i.e. a log₂ fold change in median expression of −0.36 (Imig et al., (2015). miR-CLIP capture of a miRNA targetome uncovers a lincRNA H19-miR-106a interaction. Nat Chem Biol 11, 107-114).

The seven targetomes (let-7, miRs-9, -1, -29, -30, -107, and miR-181) showed generally lower levels of expression in HV cells compared to in DM1 and DM2 myoblasts (FIGS. 6B and 6D). From myotubes, lower amounts of RNA were generally obtained from the DM1 and DM2 samples compared to those from healthy volunteers (and myoblasts in general). Without wishing to be bound by theory, this is likely due to a heterogeneous differentiation between individual patient samples. Treatment of DM1 myoblasts with Compound II produced a statistically significant suppression of the targetomes of let-7 and miRs -9, -1, -29, -30, -107, -181 with a median suppression range of 10-12% (FIG. 5A). A similar outcome was found for the treatment of DM2 myoblasts for the seven targetomes (22-25% median suppression) (FIG. 5C). The comparatively small number of miR-122 targets were not significantly changed by Compound II treatment in DM1 myoblasts, and showed a weaker change in DM2 myoblasts compared to targetomes of other miRNAs. Without wishing to be bound by theory, the degree of suppression of these targetomes resulting from Compound II treatment appeared to be consistent with the reciprocal increases in endogenous miRNA levels of 1.5-3.0-fold (FIG. 4A). In myotubes, the Compound II treatments of DM1 and DM2 samples produced equally robust effects on the seven miRNA targetomes. For DM1, the median suppression was 12-15%, whereas it reached 10-12% for DM2 samples (FIGS. 6A and 6C). miR-122 targets were not significantly changed by Compound II treatment in DM1 and DM2 myotubes.

As shown in FIGS. 5B, 5D, 6B and 6D, treatment with Compound II shifted the targetomes of the eight miRNAs tested to levels comparable to those of healthy volunteers. For DM1 myoblasts, Compound II-treatments shifted the DM1 curve towards that of the HV signature (FIG. 5B); for miR-1, miR-9, miR-29, miR-30 and miR-107, the Compound II treatment of the DM1 myoblasts produced an indistinguishable overlay in the CDF curves of the DM1+Compound II and the HV targetome signature. For DM2 myoblasts, Compound II treatment caused an “overshoot” (FIG. 5D), suggesting without wishing to be bound by theory that a lower Compound II concentration would suffice for the same disease-to-healthy “conversion”. Without wishing to be bound by theory, the data suggest that the inventive compounds can revert a DM1/2 signature to a healthy signature by increasing the activities of a subset of Lin28- and MEF-regulated miRNAs.

In one aspect, the present invention provides Compound I and/or Compound II, or a pharmaceutically acceptable salt, stereoisomer, enantiomer, tautomer, hydrate or solvate thereof, for use in a method of treating a myotonic dystrophy in a subject.

In one aspect, the present invention provides Compound I, or a pharmaceutically acceptable salt, stereoisomer, enantiomer, tautomer, hydrate or solvate thereof, for use in a method of treating a myotonic dystrophy in a subject.

In one aspect, the present invention provides Compound II, or a pharmaceutically acceptable salt, stereoisomer, enantiomer, tautomer, hydrate or solvate thereof, for use in a method of treating a myotonic dystrophy in a subject.

In one aspect, the present invention provides a method of treating a myotonic dystrophy in a subject in need thereof, the method comprising administering to the subject an effective amount of Compound I and/or Compound II, or a pharmaceutically acceptable salt, stereoisomer, enantiomer, tautomer, hydrate or solvate thereof.

In some embodiments, the present invention provides a method of treating a myotonic dystrophy in a subject in need thereof, the method comprising administering to the subject an effective amount of Compound I, or a pharmaceutically acceptable salt, stereoisomer, enantiomer, tautomer, hydrate or solvate thereof.

In some embodiments, the present invention provides a method of treating a myotonic dystrophy in a subject in need thereof, the method comprising administering to the subject an effective amount of Compound II, or a pharmaceutically acceptable salt, stereoisomer, enantiomer, tautomer, hydrate or solvate thereof.

In one aspect, the present invention provides the use of Compound I and/or Compound II, or a pharmaceutically acceptable salt, stereoisomer, enantiomer, tautomer, hydrate or solvate thereof, in the manufacture of a medicament for preventing or treating a myotonic dystrophy in a subject.

In one aspect, the present invention provides the use of Compound I, or a pharmaceutically acceptable salt, stereoisomer, enantiomer, tautomer, hydrate or solvate thereof, in the manufacture of a medicament for preventing or treating a myotonic dystrophy in a subject.

In one aspect, the present invention provides the use of Compound II, or a pharmaceutically acceptable salt, stereoisomer, enantiomer, tautomer, hydrate or solvate thereof, in the manufacture of a medicament for preventing or treating a myotonic dystrophy in a subject.

In some embodiments, the myotonic dystrophy is myotonic dystrophy type 1. In some embodiments, the myotonic dystrophy is myotonic dystrophy type 1, wherein said myotonic dystrophy type 1 is selected from congenital (CDM1), infantile, juvenile, adult, and late-onset DM1. In some embodiments, the myotonic dystrophy is congenital myotonic dystrophy type 1 (CDM1).

In some embodiments, the myotonic dystrophy is myotonic dystrophy type 2.

In some embodiments, said preventing or treating a myotonic dystrophy in a subject comprises inhibiting Lin28 activity.

In some embodiments, the present invention provides Compound I and/or Compound II for use in inhibiting Lin28 activity in a subject in a subject, preferably wherein said subject has myotonic dystrophy.

In some embodiments, the present invention provides Compound I and/or Compound II for use in a method of inhibiting Lin28 activity in a subject, preferably wherein said subject has myotonic dystrophy.

In some preferred embodiments, the present invention provides Compound I and/or Compound II for use in inhibiting Lin28A and/or Lin28B activity.

In some preferred embodiments, the present invention provides Compound I and/or Compound II for use in inhibiting Lin28A activity.

In some preferred embodiments, the present invention provides Compound I and/or Compound II for use in inhibiting Lin28B activity.

In some embodiments, the present invention provides a method of inhibiting Lin28 activity (e.g., Lin28A and/or Lin28B activity) in a subject in need thereof, the method comprising administering to said subject an effective amount of a Compound I and/or Compound II or a pharmaceutically acceptable salt, stereoisomer, enantiomer, tautomer, hydrate, or solvate thereof.

In some embodiments, the present invention provides a method of inhibiting Lin28A activity in a subject in need thereof, the method comprising administering to said subject an effective amount of a Compound I and/or Compound II or a pharmaceutically acceptable salt, stereoisomer, enantiomer, tautomer, hydrate, or solvate thereof.

In some embodiments, the present invention provides a method of inhibiting Lin28B activity in a subject in need thereof, the method comprising administering to said subject an effective amount of a Compound I and/or Compound II or a pharmaceutically acceptable salt, stereoisomer, enantiomer, tautomer, hydrate, or solvate thereof.

In some embodiments, the present disclosure provides the use of a Compound I and/or Compound II or a pharmaceutically acceptable salt, stereoisomer, enantiomer, tautomer, hydrate, or solvate thereof, in the manufacture of a medicament for inhibiting Lin28 activity, e.g., Lin28A and/or Lin28B activity).

In some embodiments, the present disclosure provides the use of a Compound I and/or Compound II or a pharmaceutically acceptable salt, stereoisomer, enantiomer, tautomer, hydrate, or solvate thereof, in the manufacture of a medicament for inhibiting Lin28A activity.

In some embodiments, the present disclosure provides the use of a Compound I and/or Compound II or a pharmaceutically acceptable salt, stereoisomer, enantiomer, tautomer, hydrate, or solvate thereof, in the manufacture of a medicament for inhibiting Lin28B activity.

In some embodiments, the Compound I and/or Compound II inhibit Lin28 (e.g., Lin28A and/or Lin28B) activity, preferably wherein said Lin28 activity is decreased by about 10%; more preferably by about 20%; more preferably by about 30%; more preferably by about 40%; more preferably by about 50%; more preferably by about 60%; more preferably by about 70%, compared to pre-treatment levels. In preferred embodiments, said Lin28 (e.g., Lin28A and/or Lin28B) activity is decreased by about 50% to about 65% compared to pre-treatment levels.

In some embodiments, said preventing or treating a myotonic dystrophy in a subject comprises increasing MEF2 expression.

In some embodiments, the present invention provides Compound I and/or Compound II or a pharmaceutically acceptable salt, stereoisomer, enantiomer, tautomer, hydrate, or solvate thereof, for use in increasing MEF2 expression, preferably MEF2A expression, in a subject, preferably wherein said subject has myotonic dystrophy.

In some embodiments, the present invention provides Compound I and/or Compound II or a pharmaceutically acceptable salt, stereoisomer, enantiomer, tautomer, hydrate, or solvate thereof, for use in a method of increasing MEF2 expression, preferably MEF2A expression, in a subject, preferably wherein said subject has myotonic dystrophy.

In some embodiments, the present invention provides a method of increasing MEF2 expression, preferably MEF2A expression, in a subject in need thereof, the method comprising administering to said subject an effective amount of Compound I and/or Compound II, or a pharmaceutically acceptable salt, stereoisomer, enantiomer, tautomer, hydrate, or solvate thereof. In preferred embodiments, said subject has myotonic dystrophy.

In some embodiments, the present invention provides the use of a Compound I and/or Compound II or a pharmaceutically acceptable salt, stereoisomer, enantiomer, tautomer, hydrate, or solvate thereof, in the manufacture of a medicament for increasing MEF2, preferably MEF2A expression, in a subject in need thereof. In preferred embodiments, said subject has myotonic dystrophy.

In some embodiments, Compound I and/or Compound II increase MEF2, preferably MEF2A expression, and preferably wherein said expression is increased by about 150%; more preferably by about 175%; more preferably by about 200%; more preferably by about 225%; more preferably by about 250%; more preferably by about 275%; more preferably by about 300%; compared to pre-treatment levels.

In some embodiments, Compound I increases MEF2, preferably MEF2A expression, and preferably wherein said expression is increased by about 150%; more preferably by about 175%; more preferably by about 200%; more preferably by about 225%; more preferably by about 250%; more preferably by about 275%; more preferably by about 300%; compared to pre-treatment levels.

In some embodiments, Compound II increases MEF2, preferably MEF2A expression, and preferably wherein said expression is increased by about 150%; more preferably by about 175%; more preferably by about 200%; more preferably by about 225%; more preferably by about 250%; more preferably by about 275%; more preferably by about 300%; compared to pre-treatment levels.

In some embodiments, said preventing or treating a myotonic dystrophy in a subject comprises increasing miRNA expression.

In some embodiments, the present invention provides a Compound I and/or Compound II or a pharmaceutically acceptable salt, stereoisomer, enantiomer, tautomer, hydrate, or solvate thereof, for use in increasing miRNA expression in a subject, preferably wherein said subject has myotonic dystrophy.

In some embodiments, the present invention provides a Compound I and/or Compound II or a pharmaceutically acceptable salt, stereoisomer, enantiomer, tautomer, hydrate, or solvate thereof, for use in a method of increasing miRNA expression in a subject, preferably wherein said subject has myotonic dystrophy.

In some embodiments, the present invention provides a method of increasing miRNA expression in a subject in need thereof, the method comprising administering to said subject an effective amount of a Compound I and/or Compound II, or a pharmaceutically acceptable salt, stereoisomer, enantiomer, tautomer, hydrate, or solvate thereof. In preferred embodiments, said subject has myotonic dystrophy.

In some embodiments, the present invention provides the use of a Compound I and/or Compound II or a pharmaceutically acceptable salt, stereoisomer, enantiomer, tautomer, hydrate, or solvate thereof, in the manufacture of a medicament for increasing miRNA in a subject in need thereof. In preferred embodiments, said subject has myotonic dystrophy.

In preferred embodiments, the Compound I and/or Compound II increase expression of let7i; miR-1; miR-9; miR-29a; miR-30a; miR-30c; miR-107; miR-133; miR-181a; miR-181b; and/or let-7g.

In preferred embodiments, Compound I and/or Compound II increase the expression of let7i. Preferably said let7i is increased by about 150%, more preferably by about 200%, more preferably by about 250%, yet more preferably by about 300% compared to pre-treatment levels.

In preferred embodiments, Compound I and/or Compound II increase the expression of miR-1. Preferably said miR-1 is increased by about 150%, more preferably by about 200%, more preferably by about 250%, yet more preferably by about 300% compared to pre-treatment levels.

In preferred embodiments, Compound I and/or Compound II increase the expression of miR-9. Preferably said miR-9 is increased by about 150%, more preferably by about 200%, more preferably by about 250%, yet more preferably by about 300% compared to pre-treatment levels.

In preferred embodiments, Compound I and/or Compound II increase the expression of miR-29a. Preferably said miR-29a is increased by about 150%, more preferably by about 200%, more preferably by about 250%, yet more preferably by about 300% compared to pre-treatment levels.

In preferred embodiments, Compound I and/or Compound II increase the expression of miR-30a. Preferably said miR-30a is increased by about 150%, more preferably by about 200%, more preferably by about 250%, yet more preferably by about 300% compared to pre-treatment levels.

In preferred embodiments, Compound I and/or Compound II increase the expression of miR-30c. Preferably said miR-30c is increased by about 150%, more preferably by about 200%, more preferably by about 250%, yet more preferably by about 300% compared to pre-treatment levels.

In preferred embodiments, Compound I and/or Compound II increase the expression of miR-107. Preferably said miR-107 is increased by about 150%, more preferably by about 200%, more preferably by about 250%, yet more preferably by about 300% compared to pre-treatment levels.

In preferred embodiments, Compound I and/or Compound II increase the expression of miR-133. Preferably said miR-133 is increased by about 150%, more preferably by about 200%, more preferably by about 250%, yet more preferably by about 300% compared to pre-treatment levels.

In preferred embodiments, Compound I and/or Compound II increase the expression of miR-181a. Preferably said miR-181a is increased by about 150%, more preferably by about 200%, more preferably by about 250%, yet more preferably by about 300% compared to pre-treatment levels.

In preferred embodiments, Compound I and/or Compound II increase the expression of miR-181b. Preferably said miR-181b is increased by about 150%, more preferably by about 200%, more preferably by about 250%, yet more preferably by about 300% compared to pre-treatment levels.

In preferred embodiments, Compound I and/or Compound II increase the expression of let-7g. Preferably said let-7g is increased by about 150%, more preferably by about 200%, more preferably by about 250%, yet more preferably by about 300% compared to pre-treatment levels.

In some embodiments, said preventing or treating a myotonic dystrophy in a subject comprises decreasing ion channel expression.

In some embodiments, the present invention provides Compound I and/or Compound II or a pharmaceutically acceptable salt, stereoisomer, enantiomer, tautomer, hydrate, or solvate thereof, for use in decreasing ion channel expression in a subject, preferably wherein said ion channel is associated with DM (preferably DM1 and/or DM2), more preferably wherein said subject has myotonic dystrophy.

In some embodiments, the present invention provides Compound I and/or Compound II or a pharmaceutically acceptable salt, stereoisomer, enantiomer, tautomer, hydrate, or solvate thereof, for use in a method of decreasing ion channel expression in a subject, preferably wherein said ion channel is associated with DM (preferably DM1 and/or DM2), more preferably wherein said subject has myotonic dystrophy.

In some embodiments, the present invention provides a method of decreasing ion channel expression in a subject in need thereof, the method comprising administering to said subject an effective amount of Compound I and/or Compound II, or a pharmaceutically acceptable salt, stereoisomer, enantiomer, tautomer, hydrate, or solvate thereof. In preferred embodiments, said subject has myotonic dystrophy.

In some embodiments, the present invention provides the use of a Compound I and/or Compound II, or a pharmaceutically acceptable salt, stereoisomer, enantiomer, tautomer, hydrate, or solvate thereof, in the manufacture of a medicament for decreasing ion channel expression in a subject in need thereof. In preferred embodiments, said subject has myotonic dystrophy.

In some embodiments, Compound I and/or Compound II decrease ion channel expression in a subject, wherein said ion channel is ATP1B1, SERCA2, KCNJ2, CACNA1S, SERCA1, and/or CACNA1C. In preferred embodiments, said ion channel expression is reduced by about 30%; preferably by about 35%; more preferably by about 40%; more preferably by about 45%; more preferably by about 50%; more preferably by about 55%; more preferably by about 60%; more preferably by about 65%; more preferably by about 70%; more preferably by about 75%; more preferably by about 80%; more preferably by about 85%; more preferably by about 90%; more preferably by about 95%; more preferably by about 99%, compared to pre-treatment levels.

In some embodiments, Compound I and/or Compound II decrease ion channel expression, wherein said ion channel is ATP1B1. In preferred embodiments, said ATP1B1 expression is reduced by about 30%; preferably by about 35%; more preferably by about 40%; more preferably by about 45%; more preferably by about 50%; more preferably by about 55%; more preferably by about 60%; more preferably by about 65%; more preferably by about 70%; more preferably by about 75%; more preferably by about 80%; more preferably by about 85%; more preferably by about 90%; more preferably by about 95%; more preferably by about 99%, compared to pre-treatment levels.

In some embodiments, Compound I and/or Compound II decrease ion channel expression, wherein said ion channel is SERCA2. In preferred embodiments, said SERCA2 expression is reduced by about 30%; preferably by about 35%; more preferably by about 40%; more preferably by about 45%; more preferably by about 50%; more preferably by about 55%; more preferably by about 60%; more preferably by about 65%; more preferably by about 70%; more preferably by about 75%; more preferably by about 80%; more preferably by about 85%; more preferably by about 90%; more preferably by about 95%; more preferably by about 99%, compared to pre-treatment levels.

In some embodiments, Compound I and/or Compound II decrease ion channel expression, wherein said ion channel is KCNJ2. In preferred embodiments, said KCNJ2 expression is reduced by about 30%; preferably by about 35%; more preferably by about 40%; more preferably by about 45%; more preferably by about 50%; more preferably by about 55%; more preferably by about 60%; more preferably by about 65%; more preferably by about 70%; more preferably by about 75%; more preferably by about 80%; more preferably by about 85%; more preferably by about 90%; more preferably by about 95%; more preferably by about 99%, compared to pre-treatment levels.

In some embodiments, Compound I and/or Compound II decrease ion channel expression, wherein said ion channel is CACNA1S. In preferred embodiments, said CACNA1S expression is reduced by about 30%; preferably by about 35%; more preferably by about 40%; more preferably by about 45%; more preferably by about 50%; more preferably by about 55%; more preferably by about 60%; more preferably by about 65%; more preferably by about 70%; more preferably by about 75%; more preferably by about 80%; more preferably by about 85%; more preferably by about 90%; more preferably by about 95%; more preferably by about 99%, compared to pre-treatment levels.

In some embodiments, Compound I and/or Compound II decrease ion channel expression, wherein said ion channel is SERCA1. In preferred embodiments, said SERCA1 expression is reduced by about 30%; preferably by about 35%; more preferably by about 40%; more preferably by about 45%; more preferably by about 50%; more preferably by about 55%; more preferably by about 60%; more preferably by about 65%; more preferably by about 70%; more preferably by about 75%; more preferably by about 80%; more preferably by about 85%; more preferably by about 90%; more preferably by about 95%; more preferably by about 99%, compared to pre-treatment levels.

In some embodiments, Compound I and/or Compound II decrease ion channel expression, wherein said ion channel is CACNA1C. In preferred embodiments, said CACNA1C expression is reduced by about 30%; preferably by about 35%; more preferably by about 40%; more preferably by about 45%; more preferably by about 50%; more preferably by about 55%; more preferably by about 60%; more preferably by about 65%; more preferably by about 70%; more preferably by about 75%; more preferably by about 80%; more preferably by about 85%; more preferably by about 90%; more preferably by about 95%; more preferably by about 99%, compared to pre-treatment levels.

In some embodiments, said preventing or treating a myotonic dystrophy in a subject comprises decreasing CELF1 expression.

In some embodiments, the present invention provides Compound I and/or Compound II, or a pharmaceutically acceptable salt, stereoisomer, enantiomer, tautomer, hydrate, or solvate thereof, for use in decreasing CELF1 expression in a subject, preferably wherein said subject has myotonic dystrophy.

In some embodiments, the present invention provides Compound I and/or Compound II, or a pharmaceutically acceptable salt, stereoisomer, enantiomer, tautomer, hydrate, or solvate thereof, for use in a method of decreasing CELF1 expression in a subject, preferably wherein said subject has myotonic dystrophy.

In some embodiments, the present invention provides a method of decreasing CELF1 expression in a subject in need thereof, the method comprising administering to said subject an effective amount of Compound I and/or Compound II, or a pharmaceutically acceptable salt, stereoisomer, enantiomer, tautomer, hydrate, or solvate thereof. In preferred embodiments, said subject has myotonic dystrophy.

In some embodiments, the present invention provides the use of Compound I and/or Compound II, or a pharmaceutically acceptable salt, stereoisomer, enantiomer, tautomer, hydrate, or solvate thereof, in the manufacture of a medicament for decreasing CELF1 expression in a subject. In preferred embodiments, said subject has myotonic dystrophy.

In some embodiments, said preventing or treating a myotonic dystrophy in a subject comprises increasing expression of CLCN1.

In some embodiments, the present invention provides Compound I and/or Compound II, or a pharmaceutically acceptable salt, stereoisomer, enantiomer, tautomer, hydrate, or solvate thereof, for use in increasing CLCN1 expression in a subject, preferably wherein said subject has myotonic dystrophy.

In some embodiments, the present invention provides Compound I and/or Compound II, or a pharmaceutically acceptable salt, stereoisomer, enantiomer, tautomer, hydrate, or solvate thereof, for use in a method of increasing CLCN1 expression in a subject, preferably wherein said subject has myotonic dystrophy.

In some embodiments, the present invention provides a method of increasing expression of CLCN1 in a subject in need thereof, the method comprising administering to said subject an effective amount of Compound I and/or Compound II, or a pharmaceutically acceptable salt, stereoisomer, enantiomer, tautomer, hydrate, or solvate thereof. In preferred embodiments, said subject has myotonic dystrophy.

In some embodiments, the present invention provides the use of Compound I and/or Compound II, or a pharmaceutically acceptable salt, stereoisomer, enantiomer, tautomer, hydrate, or solvate thereof, in the manufacture of a medicament for increasing expression of CLCN1 in a subject in need thereof. In preferred embodiments, said subject has myotonic dystrophy.

In preferred embodiments, the inventive compounds increase CLCN1 expression (e.g., in a subject) to about 110% relative to controls, preferably to about 120%, more preferably to about 130%, more preferably to about 140%, more preferably to about 150% relative to controls.

In some embodiments, said preventing or treating a myotonic dystrophy in a subject comprises decreasing expression of PKC.

In some embodiments, the present invention provides Compound I and/or Compound II, or a pharmaceutically acceptable salt, stereoisomer, enantiomer, tautomer, hydrate, or solvate thereof, for use in decreasing PKC expression in a subject, preferably wherein said subject has myotonic dystrophy.

In some embodiments, the present invention provides Compound I and/or Compound II, or a pharmaceutically acceptable salt, stereoisomer, enantiomer, tautomer, hydrate, or solvate thereof, for use in a method of decreasing PKC expression in a subject, preferably wherein said subject has myotonic dystrophy.

In some embodiments, the present invention provides a method of decreasing expression of PKC in a subject in need thereof, the method comprising administering to said subject an effective amount of Compound I and/or Compound II, or a pharmaceutically acceptable salt, stereoisomer, enantiomer, tautomer, hydrate, or solvate thereof. In preferred embodiments, said subject has myotonic dystrophy.

In some embodiments, the present invention provides the use of Compound I and/or Compound II, or a pharmaceutically acceptable salt, stereoisomer, enantiomer, tautomer, hydrate, or solvate thereof, in the manufacture of a medicament for decreasing PKC expression in a subject in need thereof. In preferred embodiments, said subject has myotonic dystrophy.

In preferred embodiments, said decreasing PKC expression comprises decreasing PKCα expression. In preferred embodiments, said decreasing PKC expression comprises decreasing p-PKCα expression. In preferred embodiments, said PKC expression is reduced by about 10%, preferably about 20%, more preferably about 30%, more preferably about 40%, more preferably about 50%.

In some embodiments, said preventing or treating a myotonic dystrophy in a subject comprises (i) inhibiting Lin28 activity; increasing MEF2 expression; (iii) increasing miRNA expression; (iv) decreasing ion channel expression; (v) decreasing CELF1 expression; increasing CLCN1 expression; and/or (vi) decreasing PKC expression in said subject.

In some embodiments, the present invention provides Compound I and/or Compound II, or a pharmaceutically acceptable salt, stereoisomer, enantiomer, tautomer, hydrate, or solvate thereof, for use in (i) inhibiting Lin28 activity; increasing MEF2 expression; (iii) increasing miRNA expression; (iv) decreasing ion channel expression; (v) decreasing CELF1 expression; increasing CLCN1 expression; and/or (vi) decreasing PKC expression in a subject in need thereof, preferably wherein said subject has DM.

In some embodiments, the present invention provides Compound I and/or Compound II, or a pharmaceutically acceptable salt, stereoisomer, enantiomer, tautomer, hydrate, or solvate thereof, for use in a method of (i) inhibiting Lin28 activity; increasing MEF2 expression; (iii) increasing miRNA expression; (iv) decreasing ion channel expression; (v) decreasing CELF1 expression; increasing CLCN1 expression; and/or (vi) decreasing PKC expression in a subject in need thereof, preferably wherein said subject has DM.

In some embodiments, the present invention provides a method of (i) inhibiting Lin28 activity; increasing MEF2 expression; (iii) increasing miRNA expression; (iv) decreasing ion channel expression; (v) decreasing CELF1 expression; increasing CLCN1 expression; and/or (vi) decreasing PKC expression in a subject in need thereof, the method comprising administering to said subject an effective amount of Compound I and/or Compound II, or a pharmaceutically acceptable salt, stereoisomer, enantiomer, tautomer, hydrate, or solvate thereof. In preferred embodiments, said subject has myotonic dystrophy.

In some embodiments, the present invention provides the use of Compound I and/or Compound II, or a pharmaceutically acceptable salt, stereoisomer, enantiomer, tautomer, hydrate, or solvate thereof, in the manufacture of a medicament for (i) inhibiting Lin28 activity; increasing MEF2 expression; (iii) increasing miRNA expression; (iv) decreasing ion channel expression; (v) decreasing CELF1 expression; increasing CLCN1 expression; and/or (vi) decreasing PKC expression in a subject in need thereof. In preferred embodiments, said subject has myotonic dystrophy.

In some embodiments, said preventing or treating a myotonic dystrophy in a subject comprises reducing muscle atrophy, reducing muscle myotonia, increasing muscle strength, reducing progressive wasting, reducing fibrosis, reducing myofiber atrophy, and/or reducing cardiac dysfunction.

In one aspect, the present invention provides Compound I and/or Compound II, or a pharmaceutically acceptable salt, stereoisomer, enantiomer, tautomer, hydrate or solvate thereof, for use in a method of reducing muscle atrophy in a subject. In preferred embodiments, said subject has myotonic dystrophy.

In one aspect, the present invention provides a method of reducing muscle atrophy in a subject in need thereof, the method comprising administering to said subject an effective amount of Compound I and/or Compound II, or a pharmaceutically acceptable salt, stereoisomer, enantiomer, tautomer, hydrate, or solvate thereof. In preferred embodiments, said subject has myotonic dystrophy.

In one aspect, the present invention provides the use of Compound I and/or Compound II, or a pharmaceutically acceptable salt, stereoisomer, enantiomer, tautomer, hydrate, or solvate thereof, in the manufacture of a medicament for reducing muscle atrophy in a subject. In preferred embodiments, said subject has myotonic dystrophy.

In one aspect, the present invention provides Compound I and/or Compound II, or a pharmaceutically acceptable salt, stereoisomer, enantiomer, tautomer, hydrate or solvate thereof, for use in a method of reducing muscle myotonia in a subject. In preferred embodiments, said subject has myotonic dystrophy.

In one aspect, the present invention provides a method of reducing muscle myotonia in a subject in need thereof, the method comprising administering to said subject an effective amount of Compound I and/or Compound II, or a pharmaceutically acceptable salt, stereoisomer, enantiomer, tautomer, hydrate, or solvate thereof. In preferred embodiments, said subject has myotonic dystrophy.

In one aspect, the present invention provides the use of Compound I and/or Compound II, or a pharmaceutically acceptable salt, stereoisomer, enantiomer, tautomer, hydrate, or solvate thereof, in the manufacture of a medicament for reducing muscle myotonia in a subject.

In preferred embodiments, said subject has myotonic dystrophy.

In one aspect, the present invention provides Compound I and/or Compound II, or a pharmaceutically acceptable salt, stereoisomer, enantiomer, tautomer, hydrate or solvate thereof, for use in a method of increasing muscle strength in a subject. In preferred embodiments, said subject has myotonic dystrophy.

In one aspect, the present invention provides a method of increasing muscle strength in a subject in need thereof, the method comprising administering to said subject an effective amount of Compound I and/or Compound II, or a pharmaceutically acceptable salt, stereoisomer, enantiomer, tautomer, hydrate, or solvate thereof. In preferred embodiments, said subject has myotonic dystrophy.

In one aspect, the present invention provides the use of Compound I and/or Compound II, or a pharmaceutically acceptable salt, stereoisomer, enantiomer, tautomer, hydrate, or solvate thereof, in the manufacture of a medicament for increasing muscle strength in a subject. In preferred embodiments, said subject has myotonic dystrophy.

In one aspect, the present invention provides Compound I and/or Compound II, or a pharmaceutically acceptable salt, stereoisomer, enantiomer, tautomer, hydrate or solvate thereof, for use in a method of reducing progressive wasting in a subject. In preferred embodiments, said subject has myotonic dystrophy.

In one aspect, the present invention provides a method of reducing progressive wasting in a subject in need thereof, the method comprising administering to said subject an effective amount of Compound I and/or Compound II, or a pharmaceutically acceptable salt, stereoisomer, enantiomer, tautomer, hydrate, or solvate thereof. In preferred embodiments, said subject has myotonic dystrophy.

In one aspect, the present invention provides the use of Compound I and/or Compound II, or a pharmaceutically acceptable salt, stereoisomer, enantiomer, tautomer, hydrate, or solvate thereof, in the manufacture of a medicament for reducing progressive wasting in a subject. In preferred embodiments, said subject has myotonic dystrophy.

In one aspect, the present invention provides Compound I and/or Compound II, or a pharmaceutically acceptable salt, stereoisomer, enantiomer, tautomer, hydrate or solvate thereof, for use in a method of reducing fibrosis in a subject. In preferred embodiments, said subject has myotonic dystrophy.

In one aspect, the present invention provides a method of reducing fibrosis in a subject in need thereof, the method comprising administering to said subject an effective amount of Compound I and/or Compound II, or a pharmaceutically acceptable salt, stereoisomer, enantiomer, tautomer, hydrate, or solvate thereof. In preferred embodiments, said subject has myotonic dystrophy.

In one aspect, the present invention provides the use of Compound I and/or Compound II, or a pharmaceutically acceptable salt, stereoisomer, enantiomer, tautomer, hydrate, or solvate thereof, in the manufacture of a medicament for reducing fibrosis in a subject. In preferred embodiments, said subject has myotonic dystrophy. Preferably reducing fibrosis comprises reducing muscle fibrosis.

In one aspect, the present invention provides Compound I and/or Compound II, or a pharmaceutically acceptable salt, stereoisomer, enantiomer, tautomer, hydrate or solvate thereof, for use in a method of reducing myofiber atrophy in a subject. In preferred embodiments, said subject has myotonic dystrophy.

In one aspect, the present invention provides a method of reducing myofiber atrophy in a subject in need thereof, the method comprising administering to said subject an effective amount of Compound I and/or Compound II, or a pharmaceutically acceptable salt, stereoisomer, enantiomer, tautomer, hydrate, or solvate thereof. In preferred embodiments, said subject has myotonic dystrophy.

In one aspect, the present invention provides the use of Compound I and/or Compound II, or a pharmaceutically acceptable salt, stereoisomer, enantiomer, tautomer, hydrate, or solvate thereof, in the manufacture of a medicament for reducing myofiber atrophy in a subject. In preferred embodiments, said subject has myotonic dystrophy.

In one aspect, the present invention provides Compound I and/or Compound II, or a pharmaceutically acceptable salt, stereoisomer, enantiomer, tautomer, hydrate or solvate thereof, for use in a method of reducing cardiac dysfunction in a subject. In preferred embodiments, said subject has myotonic dystrophy.

In one aspect, the present invention provides a method of reducing cardiac dysfunction in a subject in need thereof, the method comprising administering to said subject an effective amount of Compound I and/or Compound II, or a pharmaceutically acceptable salt, stereoisomer, enantiomer, tautomer, hydrate, or solvate thereof. In preferred embodiments, said subject has myotonic dystrophy.

In one aspect, the present invention provides the use of Compound I and/or Compound II, or a pharmaceutically acceptable salt, stereoisomer, enantiomer, tautomer, hydrate, or solvate thereof, in the manufacture of a medicament for reducing cardiac dysfunction in a subject. In preferred embodiments, said subject has myotonic dystrophy. Preferably said cardiac dysfunction is cardiac arrhythmia or cardiomyopathy.

In one aspect, the present invention provides Compound I and/or Compound II, or a pharmaceutically acceptable salt, stereoisomer, enantiomer, tautomer, hydrate or solvate thereof, for use in a method of treating amyotrophic lateral sclerosis (ALS), e.g., for use in a method of treating amyotrophic lateral sclerosis (ALS) in a subject.

In one aspect, the present invention provides a method of treating amyotrophic lateral sclerosis (ALS) in a subject in need thereof, the method comprising administering to said subject an effective amount of Compound I and/or Compound II, or a pharmaceutically acceptable salt, stereoisomer, enantiomer, tautomer, hydrate, or solvate thereof.

In one aspect, the present invention provides the use of Compound I and/or Compound II, or a pharmaceutically acceptable salt, stereoisomer, enantiomer, tautomer, hydrate, or solvate thereof, in the manufacture of a medicament for treating amyotrophic lateral sclerosis (ALS) in a subject.

In one aspect, the present invention provides Compound I and/or Compound II, or a pharmaceutically acceptable salt, stereoisomer, enantiomer, tautomer, hydrate or solvate thereof, for use in a method of treating Huntington's disease (HD), e.g., for use in a method of treating Huntington's disease (HD) in a subject.

In one aspect, the present invention provides a method of treating Huntington's disease (HD) in a subject in need thereof, the method comprising administering to said subject an effective amount of Compound I and/or Compound II, or a pharmaceutically acceptable salt, stereoisomer, enantiomer, tautomer, hydrate, or solvate thereof.

In one aspect, the present invention provides the use of Compound I and/or Compound II, or a pharmaceutically acceptable salt, stereoisomer, enantiomer, tautomer, hydrate, or solvate thereof, in the manufacture of a medicament for treating Huntington's disease (HD) in a subject.

In one aspect, the present invention provides Compound I and/or Compound II, or a pharmaceutically acceptable salt, stereoisomer, enantiomer, tautomer, hydrate or solvate thereof, for use in a method of treating heart failure, e.g., for use in a method of treating heart failure in a subject.

In one aspect, the present invention provides a method of treating heart failure in a subject in need thereof, the method comprising administering to said subject an effective amount of Compound I and/or Compound II, or a pharmaceutically acceptable salt, stereoisomer, enantiomer, tautomer, hydrate, or solvate thereof.

In one aspect, the present invention provides the use of Compound I and/or Compound II, or a pharmaceutically acceptable salt, stereoisomer, enantiomer, tautomer, hydrate, or solvate thereof, in the manufacture of a medicament for treating heart failure in a subject.

In preferred embodiments, said heart failure is atherosclerosis.

In one aspect, the present invention provides Compound I and/or Compound II, or a pharmaceutically acceptable salt, stereoisomer, enantiomer, tautomer, hydrate or solvate thereof, for use in a method of treating bipolar disorder, e.g., for use in a method of treating bipolar disorder in a subject.

In one aspect, the present invention provides a method of treating bipolar disorder in a subject in need thereof, the method comprising administering to said subject an effective amount of Compound I and/or Compound II, or a pharmaceutically acceptable salt, stereoisomer, enantiomer, tautomer, hydrate, or solvate thereof.

In one aspect, the present invention provides the use of Compound I and/or Compound II, or a pharmaceutically acceptable salt, stereoisomer, enantiomer, tautomer, hydrate, or solvate thereof, in the manufacture of a medicament for treating bipolar disorder in a subject.

In one aspect, the present invention provides Compound I and/or Compound II, or a pharmaceutically acceptable salt, stereoisomer, enantiomer, tautomer, hydrate or solvate thereof, for use in a method of treating cancer, e.g., for use in a method of treating cancer in a subject.

In one aspect, the present invention provides a method of treating cancer in a subject in need thereof, the method comprising administering to said subject an effective amount of Compound I and/or Compound II, or a pharmaceutically acceptable salt, stereoisomer, enantiomer, tautomer, hydrate, or solvate thereof.

In one aspect, the present invention provides the use of Compound I and/or Compound II, or a pharmaceutically acceptable salt, stereoisomer, enantiomer, tautomer, hydrate, or solvate thereof, in the manufacture of a medicament for treating cancer in a subject.

In preferred embodiments, said cancer is hepatocellular carcinoma.

In one aspect, the present invention provides Compound I and/or Compound II, or a pharmaceutically acceptable salt, stereoisomer, enantiomer, tautomer, hydrate or solvate thereof, for use in a method of treating diabetes, e.g., for use in a method of treating diabetes in a subject.

In one aspect, the present invention provides a method of treating diabetes in a subject in need thereof, the method comprising administering to said subject an effective amount of Compound I and/or Compound II, or a pharmaceutically acceptable salt, stereoisomer, enantiomer, tautomer, hydrate, or solvate thereof.

In one aspect, the present invention provides the use of Compound I and/or Compound II, or a pharmaceutically acceptable salt, stereoisomer, enantiomer, tautomer, hydrate, or solvate thereof, in the manufacture of a medicament for treating diabetes in a subject.

In some embodiments, said diabetes is type 1 diabetes. In some embodiments, said diabetes is type 2 diabetes.

### EXAMPLES

### Human Primary Samples and Clinical Characterization for DM1 and DM2

Human biopsies from six patients diagnosed with DM1 or DM2 were collected and sequential evaluation of CTG and CCTG repeats were analyzed at Friedrich-Baur-Institute (Department of Neurology, LMU Kinikum), Ludwig-Maxmilians University, Munich, Germany. The authors thank Muscle Tissue Culture Collection MTCC for providing the samples. The Muscle Tissue Culture Collection is part of the German network on muscular dystrophies (MD-NET, service structure S1, 01GM0601) and the German network for mitochondrial disorders (mito-NET, project D2, 01GM0862) funded by the German ministry of education and research (BMBF, Bonn, Germany). The Muscle Tissue Culture Collection is a partner of EuroBioBank (www.eurobiobank.org) and TREAT-NMD (www.treat-nmd.eu).

### Cell Culture

Primary myoblasts were obtained from Muscle Tissue Culture Collection principal office, Friedrich-Baur-Institute, Ludwig-Maximilians University, Munich, Germany in the frame of EuroBioBank (EBB) network, in accordance with the European recommendations and material transfer agreement (MTA). Myoblasts were derived from patients suffering from myotonic dystrophy type 1 (DM1) and type 2 (DM2) with well-characterized numbers of CTG or CCTG repeats respectively and additional primary myoblasts were obtained from unaffected healthy volunteer individuals (non-DM). Primary myoblasts were cultured with skeletal muscle cell growth medium (ready to use) from PromoCell (Catalogue no. C-23060-PRO) supplemented with 1% Glutamax and 50 μg/ml Gentamicin at 37° C. with 5% CO₂. Myogenic differentiation was induced by serum deprivation by switching 90% confluent myoblast cultures to DMEM supplemented with 2% horse serum, 1% Glutamax and Pen/Strep. Immortalized myoblasts derived from an unaffected 25-year old male (Control-cl48 or Ctrl-cl48) and DM1 25-year old male patient with 2600 repeats measured in the isolated myoblasts (DM1-Cl5) were described previously (Arandel et al. (2017). Immortalized human myotonic dystrophy muscle cell lines to assess therapeutic compounds. Dis Model Mech 10, 487-497). The original biopsies were obtained from Myobank, a tissue bank affiliated to EuroBioBank with an authorization to distribute such material from the French Ministry of Higher Education and Research (authorization ref AC-2019-3502), and immortalized by MyoLine, a dedicated platform from the Centre of Research in Myology, Sorbonne Université—INSERM, Paris, France, in accordance with European recommendation. The immortalized myoblasts were proliferated and differentiated with a similar protocol following the primary myoblasts.

### Statistical Data Analysis

Unless otherwise indicated, statistical analyses were performed by using Graph Pad prism program (v7). Two-tailed student t-tests or Mann-Whitney tests, where appropriate, were used to quantify the p-values.

### Transfection

Cells were differentiated for 4 days before protein and RNA extraction. The miRNA mimics were purchased from Dharmacon (now Horizon) and were transfected at 100 nM concentration in myoblast cells at 80% confluence and 72 h after transfection the growth media was changed to differentiation medium. Cells were allowed 4 days for differentiation before harvest for protein extraction.

### Example 1

Treatment with Compounds I and II Reverses Disease Hallmarks in Samples Derived from DM1 Patients

### Small Molecule Compound I and Compound II Treatment

Primary myoblasts derived from patient samples were subjected to differentiation for 5 days. Small molecule Compound I and Compound II (synthesized according to the method described in Roos et al., 2016, A Small-Molecule Inhibitor of Lin28. ACS Chem Biol 11, 2773-2781) was solubilized in 5% DMSO and diluted with water as a stock, or only 5% DMSO with diluted water taken as control. Myoblasts were treated with appropriate concentration for 72 h before harvest for protein extraction.

### Immunoblotting

Myotubes were washed twice with PBS, then lysed at 4° C. with HEPES buffer (50 mM HEPES pH 7.4, 150 mM NaCl, 1 mM EGTA/EDTA, 10% Glycerol, 0.5% Triton X-100) and complete mini EDTA-free protease inhibitor cocktail (Roche) followed by sonication. The lysates were centrifuged and supernatants were collected for BCA quantification for protein measurement. 30-90 μg total protein per sample were resolved in a 7-10% SDS-PAGE and transferred to nitrocellulose membrane (Amersham-Protran, GE Healthcare, Life Sciences) by using wet blot (BioRad). After transfer, the nitrocellulose membranes were blocked for 1 h with 5% non-fat milk (Roth) in Tris-buffered Saline/Tween 20 (TBS-T). Thereafter, the membranes were incubated overnight with previously described primary antibodies to human LIN28A (D95F5,8706s; 1:1000, Cell Signaling), LIN28B (41965, 1:1000, Cell Signaling), MEF2A (9736S, 1:1000, Cell signaling), ATP1B1 (MA3-930 (M17-P5-F11), 1:1000, Invitrogen), SERCA1 (MA3-912 (VE121G9) 1:1000, Invitrogen), SERCA2 (95805, 1:1000, Cell signaling), CACNA1C (L57/46, ab84814, 1:1000, Abcam), CACNA1S (1A, ab2862, 1:1000, Abcam), KCNJ2 (ab109750, 1:1000), CELF1 (3B1, ab9549, 1:1000, Abcam), CLCN1, and PKCα. After washing with TBS-T, the membranes were incubated for 2 h with corresponding secondary antibodies (1:5000, Seracare-KPL). For loading control, immunoblotting was performed by using anti-GAPDH antibody (60004-1, 1:5000, Proteintech). Bands were visualized by horseradish peroxidase (HRP)-conjugated antibodies against rabbit IgG (10537462, Seracare-KPL) or mouse IgG (5220-0341, Seracare.KPL) using ECL Prime western blotting detection reagent (cytiva, Amersham) in a Biorad ChemiDoc XRS system following the manufacturer's protocol. Images were quantified using imageJ software (https://imagej.nih.gov).

The results are shown in FIGS. 1A-1D, 2A-2B, 3A-3B, 7, 8, 9, 10, 11A-11F, 12A-12F, 13A-13C, 14A-14E, 16A-16B, and 17A-17D.

### Example 2

Treatment with Compounds I and II De-Represses Mature Lin28-miRNAs

### Small Molecule Compound I and Compound II Treatment

Primary myoblasts derived from patient samples were subjected to differentiation for 5 days. Small molecule Compound I or Compound II (as obtained in Example 1) was solubilized in 5% DMSO and diluted with water as a stock, or only 5% DMSO with diluted water taken as control. Myoblasts were treated with appropriate concentration for 72 h before harvest for RNA extraction.

TaqMan Stem-Loop RT-PCR miRNA Assays

TaqMan stem-loop assays (Applied Biosystem by ThermoFisher Scientific) were carried out to quantify mature miRNA expression levels. 20 ng of total RNA from each sample was transcribed using RT primers (from ThermoFisher Scientific) and the Taqman miRNA reverse transcription kit (Applied Biosystems by ThermoFisher Scientific). qRT-PCR was performed using Tm primer (from ThermoFisher Scientific) and the GoTaq probe qPCR Master Mix (Promega) in triplicates in a 384-well plate on a Roche Light Cycler 480, following manufacturer's protocols. For normalization, U6 snRNA was used as a housekeeping gene.

Results are shown in FIGS. 4A-4B.

### Example 3

Treatment with Compounds I and II Suppresses the Targetomes of Lin28-miRNAs

### RNA Sequencing Data Generation

Primary myoblasts or myotubes derived from DM1 or DM2 patient samples and non-DM healthy control individuals were cultured for 90% confluence and then treated with 120 μM Compound II for 72 h. Myoblasts were harvested by trypsinization, and separately myotubes were isolated by a minimal trypsin treatment followed by gentle centrifugation to sediments to separate them from the remaining mono-nucleated cells. Cell pellets were washed with PBS and RNA isolation was performed with Trizol® by using the Dircet-zol™ RNA MiniPrep Plus Kit (ZYMO Research, Freiburg, Germany). Library preparation was performed using the TruSeq Stranded mRNA Kit (Illumina, San Diego, CA, USA) with the TruSeq RNA Single Index Set A (Illumina) according to the recommended procedure. Quality and size distribution of the generated libraries was validated using an Agilent 2100 bioanalyzer with high-sensitivity RNA chip (Agilent) and RNA yield was measured using the Qubit dsRNA HS Assay Kit followed by pooling of the libraries in batches of 12 and sequencing of 1.2 μM pooled library on an Nextseq 500/High Output Flow Cell Cartridge using a paired end, 2×76 reads, single index protocol.

RNA Isolation and qRT-PCR

Total RNA from primary myotubes or immortalized myotubes were isolated by using Trizol (Ambion by Life Technologies) or RNAeasy mini Kit (Qiagen), following manufacturer's protocol. First strand complementary DNA (cDNA) was reverse transcribed from mRNA (1p g) using a reverse transcriptase kit (Applied Biosystem) according to the manufacturer's protocol. Amplification of target genes was performed using corresponding primer sets (described in the supplementary information) by Sybr based quantitative real time PCR (qRT-PCR) assays (FirstStart Universal SYBR Green master, Roche Applied Sciences or KAPA SYBRFAST qPCR Master Mix) in a Roche Light Cycler 480, following manufacturer's protocols. For normalization housekeeping gene encoding glyceraldehyde 3-phosphate dehydrogenase (GAPDH) was used for normalization. mRNA values for each treatment were calculated from 3 individual samples generated in 3-4 independent experiments or from DM1 (n=6), DM2 (n=6) and non-DM healthy volunteer control (n=6) samples. Data were analyzed using Light Cycler 480 software generating cycle threshold (Ct) values and fold changes were calculated by using 2^{-ΔCt}.

Gene Expression Changes in Disease and after Drug Treatment

RNA-Seq reads were mapped to the human genome (build GRCh38) using STAR (Dobin et al., (2013). STAR: ultrafast universal RNA-seq aligner. Bioinformatics 29, 15-21) with gene annotations from gencode (www.gencodegenes.org; version 40). Reads mapping uniquely to exons were counted for each gene using htseq-count (Anders et al., (2015). HTSeq—a Python framework to work with high-throughput sequencing data. Bioinformatics 31, 166-169) with the parameter -s reverse and -m intersection_strict. Expression levels were quantified as TPM (transcripts per million), by dividing read counts by the total exon length for each gene, and then normalising by the sum of all values in each sample and multiplying by one million. Fold changes were calculated between the average expression levels in three conditions: DMSO-treated healthy volunteer samples (HV), DMSO-treated DM patient samples (DM), and Compound II-treated DM patient samples (DM+Compound II). Genes with an average TPM>1 were considered in each of these three conditions. For myoblasts, three samples were included for all conditions. For myotubes, three samples were included for the HV, DM1 and DM2 conditions, but two Compound II-treated samples were excluded (one DM1 and one DM2) due to a very low read count (<500000 exonic reads), resulting in one DM1+Compound II and two DM2+Compound II myotube samples being included in the analysis. Cumulative density functions of expression changes of miRNA target genes were plotted using custom python scripts.

Results are shown in FIGS. 5A-5D and 6A-6D.

A number of embodiments of the invention have been described. Nevertheless, it will be understood that various modifications may be made without departing from the spirit and scope of the invention. Accordingly, other embodiments are within the scope of the following claims.

1. A compound, wherein said compound is Compound I and/or Compound II, or a pharmaceutically acceptable salt, stereoisomer, enantiomer, tautomer, hydrate or solvate thereof, for use in a method of preventing or treating a myotonic dystrophy in a subject:

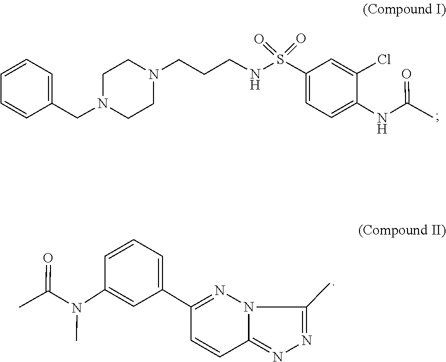

2. The compound for use according to claim 1, wherein the compound is Compound I, or a pharmaceutically acceptable salt, stereoisomer, enantiomer, tautomer, hydrate or solvate thereof.

3. The compound for use according to claim 1, wherein the compound is Compound II, or a pharmaceutically acceptable salt, stereoisomer, enantiomer, tautomer, hydrate or solvate thereof.

4. The compound for use according to any of the preceding claims, wherein the myotonic dystrophy is myotonic dystrophy type 1.

5. The compound for use according to any of claims 1-3, wherein the myotonic dystrophy is myotonic dystrophy type 2.

6. The compound for use according to any of the preceding claims, wherein said preventing or treating a myotonic dystrophy in a subject comprises inhibiting Lin28 activity.

7. The compound for use according to any of the preceding claims, wherein said preventing or treating a myotonic dystrophy in a subject comprises increasing MEF2 expression.

8. The compound for use according to any of the preceding claims, wherein said preventing or treating a myotonic dystrophy in a subject comprises increasing miRNA expression.

9. The compound for use according to any of the preceding claims, wherein said preventing or treating a myotonic dystrophy in a subject comprises decreasing ion channel expression.

10. The compound for use according to any of the preceding claims, wherein said preventing or treating a myotonic dystrophy in a subject comprises decreasing CELF1 expression.

11. The compound for use according to any of the preceding claims, wherein said preventing or treating a myotonic dystrophy in a subject comprises increasing expression of CLCN1.

12. The compound for use according to any of the preceding claims, wherein said preventing or treating a myotonic dystrophy in a subject comprises decreasing expression of PKC.

13. The compound for use according to any of the preceding claims, wherein said preventing or treating a myotonic dystrophy in a subject comprises (i) inhibiting Lin28 activity; increasing MEF2 expression; (iii) increasing miRNA expression; (iv) decreasing ion channel expression; (v) decreasing CELF1 expression; increasing CLCN1 expression; and/or (vi) decreasing PKC expression in said subject.

14. The compound for use according to any of the preceding claims, wherein said preventing or treating a myotonic dystrophy in a subject comprises reducing muscle atrophy, reducing muscle myotonia, increasing muscle strength, reducing progressive wasting, reducing fibrosis, reducing myofiber atrophy, and/or reducing cardiac dysfunction.

15. A pharmaceutical composition comprising Compound I and/or Compound II according to claim 1, and a pharmaceutically acceptable carrier or excipient.
